(19) United States
(12) Patent Application Publication
(10) Pub. No.: US 2012/0259399 A1
(43) Pub. Date: Oct. 11, 2012

(54) INTRALUMINAL SCAFFOLD SYSTEM AND USE THEREOF

(75) Inventors: Randolf Von Oepen, Aptos, CA (US); Kevin J. Ehrenreich, San Francisco, CA (US)

(73) Assignee: Abbott Laboratories, Abbott Park, IL (US)

(21) Appl. No.: 13/350,515

(22) Filed: Jan. 13, 2012

Related U.S. Application Data
(60) Provisional application No. 61/433,041, filed on Jan. 14, 2011.
(60) Provisional application No. 61/433,047, filed on Jan. 14, 2011.
(60) Provisional application No. 61/433,055, filed on Jan. 14, 2011.
(60) Provisional application No. 61/433,063, filed on Jan. 14, 2011.

(51) Int. Cl. A61F 2/84; A61F 2/82

(52) U.S. Cl. 623/1.11; 623/1.15

(57) ABSTRACT

Intraluminal scaffold assembly implantable in a body lumen of a patient to manipulate a valve of the lumen is provided. The intraluminal scaffold assembly includes an intraluminal scaffold, an elongated core member coupled with the intraluminal scaffold having a length sufficient to traverse a valve in a body lumen with the intraluminal scaffold positioned proximate the valve. The intraluminal scaffold assembly can further include one or more additional intraluminal scaffolds, a weighted element or an active element that is coupled with the elongated core member. A system including a delivery system and the intraluminal scaffold assembly, as well as methods of delivering the intraluminal scaffold assembly and using the intraluminal scaffold assembly to manipulate a valve in a body lumen, is also provided.

### CROSS REFERENCES TO RELATED APPLICATIONS

This application claims priority to U.S. Provisional Application Nos. 61/433,041, 61/433,047, 61/433,055, and 61/433,063, each of which was filed on Jan. 14, 2011, and the disclosure of each of which is hereby incorporated by reference in its entirety.

### FIELD OF DISCLOSED SUBJECT MATTER

The disclosed subject matter relates to an intraluminal scaffold assembly for deployment in a body lumen of a patient. More particularly, the disclosed subject matter relates to an intraluminal scaffold assembly and the use thereof for manipulating a venous valve.

### SUMMARY

The purpose and advantages of the disclosed subject matter will be set forth in and apparent from the description that follows, as well as will be learned by practice of the disclosed subject matter. Additional advantages of the disclosed subject matter will be realized and attained by the methods and systems particularly pointed out in the written description and claims hereof, as well as from the appended drawings.

To achieve these and other advantages and in accordance with the purpose of the disclosed subject matter, as embodied and broadly described, one aspect of the disclosed subject matter is directed to an intraluminal scaffold assembly including an intraluminal scaffold and an elongated core member coupled with the intraluminal scaffold. The elongated core member has a sufficient length to traverse the valve in a body lumen with the intraluminal scaffold positioned proximate the valve.

In some embodiments, the elongated core member includes a tail element extending laterally from the elongated core member. For example, the elongated core member can include at least two tail elements, wherein each of the at least two tail elements extends laterally from the elongated core member. The at least two tail elements can extend from a same location, or different locations, along the elongated core member. The at least two tail elements can extend in generally opposite lateral directions from each other, e.g., symmetrically with respect to the elongated core member. The at least two tail elements can have a same length or different lengths. In another embodiment, three or more tail elements can be provided, in which at least two of the at least three tail elements can extend from a same location along the elongated core member, and at least one of the at least three tail elements can extend from a second location along the elongated core member different than the first location. The tail element can have an atraumatic end to avoid or minimize trauma to the wall of the body lumen. Further, the tail element can be made of a material different than that of the remainder of the elongated core member.

In accordance with another aspect of the disclosed subject matter, the intraluminal scaffold assembly further includes an active element coupled with the elongated core member at a location spaced from the intraluminal scaffold, wherein the active element is externally actuatable to deflect the elongated core member. The active element can be magnetically active, e.g., the active element can be or include a magnet, and can be coupled at a free end of the elongated core member. In some embodiments, the intraluminal scaffold assembly further includes a source of external field, e.g., one or more magnets, which can be disposed within a housing structure, such as a pillow or a wearable article. The external field can have an adjustable strength and/or an adjustable orientation. The source of the external field can include a controller to adjust the strength and/or orientation of the external field, and/or control the external field according to a preset schedule.

In accordance with another aspect, the intraluminal scaffold assembly can include a second intraluminal scaffold coupled with the elongated core member and spaced from the first intraluminal scaffold. A portion of the elongated core member between the first intraluminal scaffold and the second intraluminal scaffold can be biased in a non-linear shape. Additionally or alternatively, a weighted element can be coupled to the elongated core member.

The intraluminal scaffold assembly can further include a second elongated core member extending from the first elongated core member at a connection location between the first intraluminal scaffold and the second intraluminal scaffold, the second elongated core member coupled with a third intraluminal scaffold. The first elongated core member and the second elongated core member can define a hinge at the connection location. Further, the intraluminal scaffold assembly can include a weighted node coupled to the elongated core member at a location between the first intraluminal scaffold and the second intraluminal scaffold. The weighted node can have a mass sufficient to cause the elongated core member to urge a portion of the valve toward an open position.

One or more intraluminal scaffolds of the intraluminal scaffold assembly described above can be a supporting scaffold, such as a stent, or alternatively, a conforming scaffold. In one example, the conforming scaffold defines a longitudinal axis and includes at least two filaments extending from a head portion disposed along the longitudinal axis at a first longitudinal end. The at least two filaments of the intraluminal scaffold can each include an end portion disposed at a second longitudinal end opposite the head portion. The at least two filaments can converge toward each other at a juncture disposed proximate the longitudinal axis between the first longitudinal end and the second longitudinal end. The end portion of each of the at least two filaments can be free, joined together, or otherwise constrained. The elongated core member can be coupled with the intraluminal scaffold at the head portion, or alternatively, the elongated core member can be coupled with the end portion of each of the at least two filaments at the second longitudinal end of the intraluminal scaffold.

In accordance with another aspect of the disclosed subject matter, an intraluminal scaffold system is provided. The system include a delivery system having an inner member having a distal end portion and an outer sheath movable relative to the inner member, the outer sheath having a first position to cover the distal end portion of the inner member and a second position to expose the distal end portion of the inner member, and an intraluminal scaffold assembly including a first intraluminal scaffold and an elongated core member coupled with the first intraluminal scaffold, wherein the elongated core member has a length sufficient to traverse a valve in a body lumen with the intraluminal scaffold positioned proximate the valve.

In accordance with a further aspect of the disclosed subject matter, a method of delivering an intraluminal scaffold assembly is provided. The method include providing an intraluminal scaffold assembly including a first intraluminal scaffold and an elongated core member coupled with the first intraluminal scaffold, and deploying the intraluminal scaffold assembly by implanting the first intraluminal scaffold at a first target site within a body lumen with the elongated core member disposed to cross a valve. The body lumen can be a blood vessel. The first target site can be in an internal jugular vein, and upstream of the valve. In addition, the first target site can be in a different blood vessel from a blood vessel housing the valve. The method can further include implanting a second intraluminal scaffold at a second target site, and/or implanting a third intraluminal scaffold at a third target site.

In an alternative method, the method includes providing an intraluminal scaffold assembly including an intraluminal scaffold, an elongated core member coupled with the intraluminal scaffold and including at least one tail element extending laterally from the elongated core member, and delivering the intraluminal scaffold assembly within a body lumen proximate a valve of the body lumen. In one embodiment, after delivery, the tail element is disposed to open the valve. The body lumen can be a vein, for example, an internal jugular vein.

In accordance with another method of the disclosed subject matter, the method includes providing an intraluminal scaffold assembly including a first intraluminal scaffold, a second intraluminal scaffold, and an elongated core member coupled with each of the first intraluminal scaffold and the second intraluminal scaffold. This method further includes implanting the first intraluminal scaffold at a first target site; implanting the second intraluminal scaffold at a second target site; and disposing the elongated core member to cross at least one valve in at least one of the left internal jugular vein or the right internal jugular vein of the patient. The first target site can be in the left internal jugular vein or left external jugular vein. The second target site can be in the right internal jugular vein or right external jugular vein. A portion of the elongated core member can be disposed to pass through a brachiocephalic vein. In one embodiment of the method, the intraluminal scaffold assembly further includes a weighted node coupled to the elongated core member at a location between the first intraluminal scaffold and the second intraluminal scaffold. The weighted node can be disposed proximate the upper end of superior vena cava.

In accordance with yet another method of the disclosed subject matter, the method includes providing an intraluminal scaffold assembly including an intraluminal scaffold, an elongated core member coupled with the intraluminal scaffold, and an active element coupled to the elongated core member and spaced from the intraluminal scaffold. This method further includes deploying the intraluminal scaffold assembly in a body lumen of a patient by implanting the intraluminal scaffold at a target site with the elongated core member disposed to cross a valve of the body lumen; and applying an external field to actuate the active element to cause a deflection of the elongated core member, thereby causing a disturbance of the valve. In this method, the body lumen can be a blood vessel, such as a vein, and more particularly, an internal jugular vein. The target site can be in a second blood vessel different from the blood vessel housing the valve. In one embodiment, the second blood vessel is the external jugular vein and target site is in an internal jugular vein. The target site can be upstream of the valve, and the intraluminal scaffold can be implanted on one side of the valve and the active element is disposed on another side of the valve. The external field can be applied when the patient is in a supine or prone position, e.g., when the patient is sleeping. Applying the external field can include controlling a strength, orientation, or a combination thereof of the external field.

### BRIEF DESCRIPTION OF THE DRAWINGS

FIG. 1 is a schematic side view of a representative embodiment of an intraluminal scaffold assembly according to the disclosed subject matter.

FIG. 2 is a schematic side view of another representative embodiment of an intraluminal scaffold assembly of the disclosed subject matter.

FIG. 3 is a schematic view the intraluminal scaffold assembly of FIG. 2 as positioned within a body lumen.

FIGS. 4A-4B are schematic views of an intraluminal scaffold assembly including a weighted element as deployed in the vasculature of a patient according to another aspect of the disclosed subject matter.

FIG. 5 is a schematic side view of a representative embodiment of an intraluminal scaffold assembly according to the disclosed subject matter.

FIGS. 6A-6C are schematic side views of different embodiments of an intraluminal scaffold assembly of the disclosed subject matter.

FIG. 7 is a schematic view of an exemplary configuration of tail elements of an intraluminal scaffold assembly of the disclosed subject matter.

FIG. 8 is a schematic view the intraluminal scaffold assembly of FIG. 6A as positioned within a body lumen.

FIG. 9 is a schematic side view of a representative embodiment of an intraluminal scaffold assembly of the disclosed subject matter.

FIG. 10 is a schematic view of the intraluminal scaffold assembly of FIG. 2 as positioned and actuated within a body lumen.

FIG. 11 is a schematic representation of an intraluminal scaffold assembly with a source of an external field housed in a pillow according to the disclosed subject matter.

FIG. 12 is a schematic representation of the use of an alternative embodiment of an intraluminal scaffold assembly as deployed in the vasculature of a patient.

FIG. 13 is an enlarged detail view illustrating manipulation of a valve using an intraluminal scaffold assembly according to the disclosed subject matter.

FIG. 14A is a schematic side view of an intraluminal scaffold assembly including two scaffolds according to another aspect of the disclosed subject matter.

FIG. 14B is a schematic view of the intraluminal scaffold assembly of FIG. 14A as deployed in the vasculature of a patient.

FIG. 15A is a schematic side view of an alternative embodiment of an intraluminal scaffold assembly as deployed in the vasculature of a patient.

FIG. 15B is a enlarged detail view of different positions of the elongated core member of the intraluminal scaffold assembly to manipulate a valve.

FIGS. 15C and 15D are schematic views of an intraluminal scaffold assembly having a biased elongated core member pressing towards a valve in different directions.

FIG. 16A is a schematic side view of an intraluminal scaffold assembly including three connected scaffolds according to another aspect of the subject matter.

FIG. 16B is a schematic side view of the intraluminal scaffold assembly of FIG. 16A as deployed in the vasculature of a patient.

FIG. 17 is a schematic front view of a representative embodiment of an intraluminal scaffold assembly of the disclosed subject matter.

FIG. 18 is a schematic front view of a portion of an alternative embodiment of an intraluminal scaffold assembly of the disclosed subject matter.

FIG. 19 is a schematic view of an intraluminal scaffold assembly as deployed in the vasculature of a patient.

FIG. 20 is a schematic view of an intraluminal scaffold assembly as deployed in an alternative configuration in the vasculature of a patient.

FIG. 21 is an enlarged view of the use of an intraluminal scaffold assembly to open a valve in the vasculature of a patient.

FIGS. 22A and 22B are schematic side views of a representative delivery system for an intraluminal scaffold assembly according to the disclosed subject matter.

FIGS. 23A and 23B are schematic diagrams of a method for delivering an intraluminal scaffold assembly including three scaffolds.

FIG. 23C is a schematic view for a delivery system including an intraluminal scaffold assembly having a pullwire wound around selected scaffolds of the assembly.

While the disclosed subject matter is capable of various modifications and alternative forms, specific embodiments thereof have been shown by way of the figures, and will herein be described in detail. It should be understood, however, that it is not intended to limit the subject matter to the particular forms disclosed but, to the contrary, the intention is to include such modifications, equivalents, and alternatives falling within the spirit and scope of the subject matter as defined by the appended claims.

### DETAILED DESCRIPTION

In accordance with one aspect of the disclosed matter, an intraluminal scaffold assembly is provided, which includes a valve manipulation element to influence or manipulate a valve of a body lumen. Such influence or manipulation can, for example, open the valve temporarily, intermittently, at a desire time(s), or according to a preset schedule, thus can help to relieve or inhibit the conditions of reduced or blocked flow through the lumen. The intraluminal scaffold assembly includes an intraluminal scaffold and an elongated core member coupled with the intraluminal scaffold. The elongated core member has a sufficient length to traverse the valve in a body lumen with the intraluminal scaffold positioned proximate the valve.

In accordance with another aspect of the disclosed subject matter, a method for deploying an intraluminal scaffold assembly is provided. The method generally includes: providing a intraluminal scaffold assembly including a first intraluminal scaffold and an elongated core member coupled with the first intraluminal scaffold, and deploying the intraluminal scaffold assembly by implanting the first intraluminal scaffold in a first target site within a body lumen with the elongated core member disposed across a valve of the body lumen. The method will be described in conjunction with the intraluminal scaffold assembly and system below.

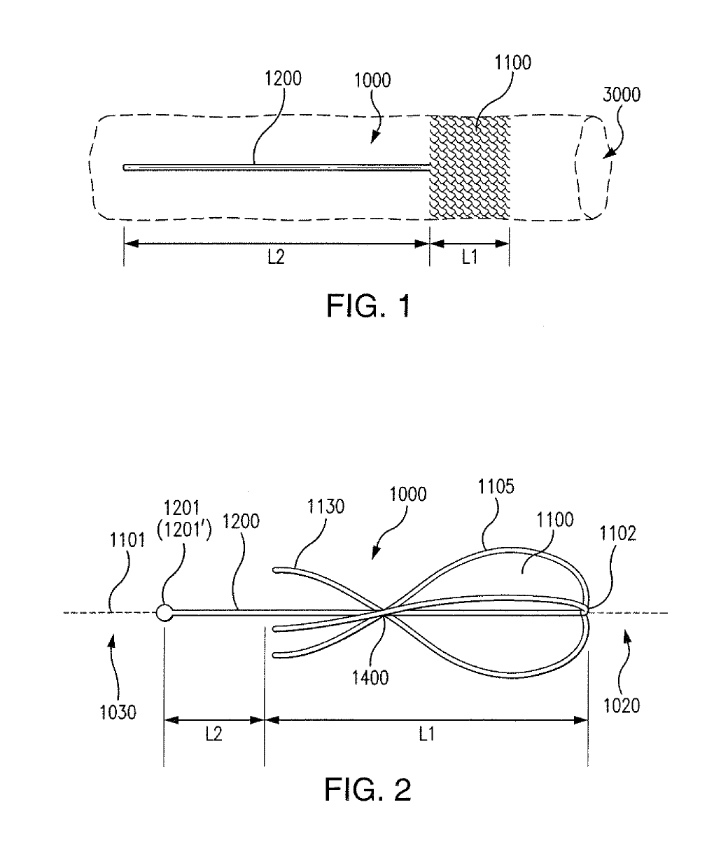

For purpose of illustration and not limitation, various embodiments of the intraluminal scaffold assembly and related delivery method are described below in conjunction with the drawings. It is noted that the figures are not to scale and certain dimensions have been exaggerated for clarity. Referring to FIG. 1, the intraluminal scaffold assembly 1000 includes an intraluminal scaffold 1100 having a longitudinal length L1. The intraluminal scaffold assembly 1000 also includes an elongated core member 1200 coupled to the scaffold 1100. The elongated core member 1200 includes a portion having a length L2 extending beyond the length L1 of the scaffold 1100 to traverse a valve in a body lumen, e.g., a blood vessel, to manipulate the valve, e.g., to temporarily open the valve.

The scaffold 1100 has an expanded profile that can engage the lumen wall of a blood vessel 3000 to provide positional stability of the intraluminal scaffold assembly in the body lumen as deployed. A variety of suitable types of intraluminal scaffolds can be used. For example, in certain circumstances, a supporting scaffold, such as a stent, spiral, anchor, or the like can be used as the scaffold in the scaffold assembly 1000. Alternatively, in certain circumstances, such as when anchoring into the lumen wall is not necessary or desired, the scaffold can be a conforming scaffold. As used herein, by “conforming scaffold”, it is intended that the overall geometry and stiffness of the scaffold is such that the scaffold can engage the lumen wall to inhibit movement within the lumen under the normal use condition without substantially altering the diameter of the lumen at its undisturbed or natural state. However, the scaffold can be suitably sized and flexible to maintain engagement with the vessel wall in response to a change in the diameter of the vessel between its smallest diameter to its maximum anticipated diameter corresponding to different physiological states of the patient. Thus, in contrast with a supporting scaffold, such as a stent for maintaining the patency of an artery, a conforming scaffold does not urge or otherwise support the lumen wall in a predetermined diameter. Rather, the conforming scaffold dynamically changes shape to adapt to the varying size of the blood vessel at different anatomical sites and in different physiological conditions, and allows for easy deployment, retrieval, and repositioning of the scaffold within the body lumen. Alternative embodiments of conforming scaffolds are disclosed and described in detail in U.S. Provisional Application 61/433,055, filed Jan. 14, 2011, which is incorporated by reference herein in its entirety.

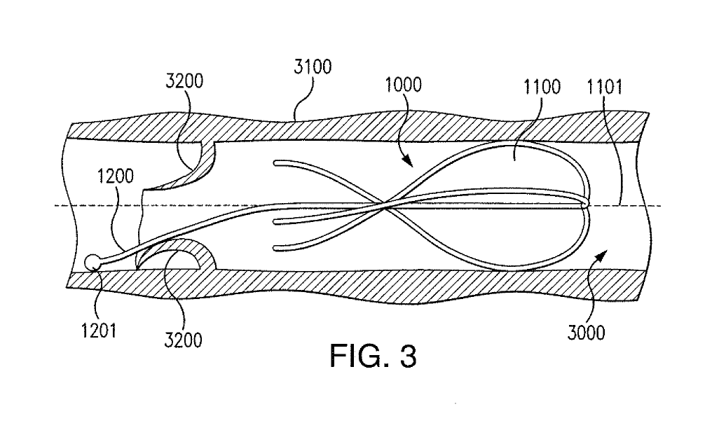

For purpose of illustration and not limitation, FIG. 2 shows a representative embodiment of a conforming scaffold, which has a longitudinal axis 1101 and at least two filaments 1105 extending from a head portion 1102 disposed along the longitudinal axis at a first longitudinal end 1020. Each of the at least two filaments 1100 includes a free end portion 1130 at a second longitudinal end 1030 that is opposite the head portion. The at least two filaments converge toward each other at a juncture 1400 disposed proximate the longitudinal axis 1101 between the first longitudinal end 1020 and the second longitudinal end 1030. The at least two filaments can converge toward each other to intersect, as shown in FIG. 2, or without intersecting each other. It is understood that the filaments can intersect or cross each other with or without actually contacting each other. In the three dimensional space, the filaments are not necessarily physically joined, welded, or otherwise constrained. For example, intersecting filaments can slide along each other when being subjected to a compression load generally perpendicular to the longitudinal axis of the scaffold.

As shown in FIG. 2, the elongated core member 1200 is coupled to the scaffold 1100 at the head portion, extends to the second longitudinal end 1030 of the scaffold 1100 and through the juncture 1400. It is appreciated, however, that the elongated core member 1200 can be configured relative to the scaffold 1100 in other manners. For example, the elongated core member can be coupled with the filaments at the juncture either alone or in addition to being coupled to the head portion. Additionally or alternatively, the length L2 of the elongated core member can extend from the head portion in a direction opposite the second end of the intraluminal scaffold. It is noted the elongated core member can be construed as a feature of the conforming scaffold and/or of the intraluminal scaffold assembly as disclosed herein if configured to manipulate the valve of a body lumen.

As shown in FIG. 1 and FIG. 2, the elongated core member generally has a smaller profile or cross dimension than the intraluminal scaffold 1100. Although the elongated core member is depicted as a straight wire member, the elongated core member can be curved or shaped as desired or needed adapt to the geometry of the body lumen in which the intraluminal scaffold assembly is implanted. The elongated core member can extend from the intraluminal scaffold along the longitudinal axis in either of both directions, and need not extend through the intraluminal scaffold as shown. The elongated core member is formed of any of a variety of suitable material, including a deformable metal such as stainless steel, a shape memory alloy such as nickel-titanium, other metals or metal alloys such as cobalt-chromium, or polymers such as nylon, PTFE, or composites as appropriate. The elongated core member can be in the form of a thin rod, ribbon, wire, or other suitable shape. The elongated core member will have a cross dimension sized to provide flexibility and to fit within the desired lumen. For example, the elongated core member can have a cross-dimension of between about 0.003 inches and 0.01 inches. The elongated core member can be constructed as a single piece, such as drawn wire, or alternatively can be formed as a cable or braided structure or the like. The length of the elongated core member will depend upon the intended application and deployment site of the intraluminal scaffold assembly. As disclosed herein, however, the length L2 of the elongated core member extend from the intraluminal scaffold is sufficiently long to enable the elongated core member to traverse the desired valve within the body lumen with the intraluminal scaffold positioned proximate the valve. A greater length will be provided if the intraluminal scaffold is to be implanted in a spaced relationship from the valve and/or if additional scaffolds are coupled with the elongated core member as described further below.

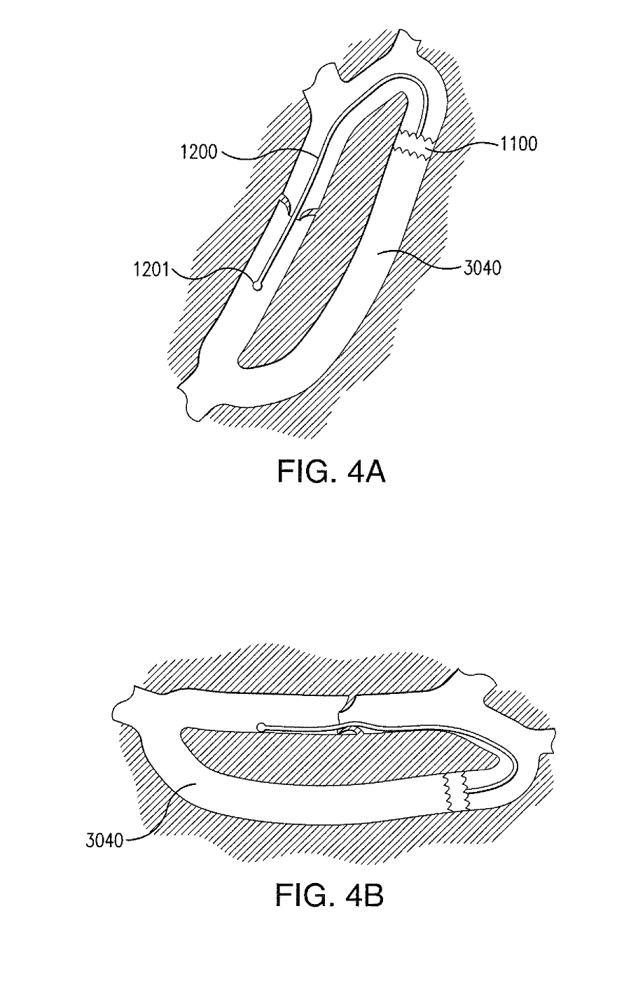

As embodied herein and as schematically illustrated in FIG. 3, the elongated core member of the intraluminal scaffold assembly can be used to manipulate a valve in the body lumen of a patient. For example, the intraluminal scaffold assembly 1000 can be implanted within a body lumen, e.g., a blood vessel 3000 having a wall 3100 with the elongated core member 1200 disposed across a valve 3200 of the blood vessel 3000. The elongated core member can thus interact with the valve, e.g., the leaflets, in a variety of manners. For example, with the elongated core member traversing and extending through the valve, the leaflets are inhibited or prevented from “sticking” or otherwise trapped in a closed position. Further, incidental movement by the patient will result in movement of the elongated core member and thus opening of the leaflets. Additionally, the elongated core member can be further configured to enhance manipulation of the valve. For example, the elongated core member can include a portion having a non-linear shape to ensure engagement with the valve. Additionally or alternatively, a weighted element can be coupled with the elongated core member, such as at the tip as shown in FIG. 3. In this manner, the weighted element can enhance manipulation of the valve by providing a greater moment applied to the elongated core member, that is, with a weighted element 1201 coupled to the tip of the elongated core member 1200 and disposed on the opposite of the valve as the intraluminal scaffold, a torque is more readily created on the elongated core member to deflect the elongated core member and contact the valve 3200. Such a torque can occur for example, by a shift in orientation or a change in flow dynamics. It is noted that FIG. 3 depicts the intraluminal scaffold implanted upstream of the valve 3200, with the elongated core member extending downstream. By contrast, the intraluminal scaffold can be implanted downstream with the elongated core member extending upstream, or the valve can be malformed so as to be inverted as shaped different than as shown.

The mass of the weighted element 1201 can be selected to be sufficient to bend the elongated core member from its tension-free position. For example, the blood vessel 3000 depicted in FIG. 3 can be an internal jugular vein of a patient so as to be aligned in a position when the patient is in a supine or prone posture, e.g., when the patient is lying horizontally. The flexibility of the elongated core member and the mass of the weighted element can be selected such that the elongated core member generally does not interfere the normal operation of the valve when the patient is in a vertical or standing posture, but urges the valve into an open configuration when the patient is in a supine, prone, or otherwise horizontal position. In this manner, the patient can receive the benefit of increased blood flow when in the horizontal position utilizing the intraluminal scaffold assembly as disclosed herein proximate the internal jugular vein.

Additionally, the intraluminal scaffold can be engaged externally for selective manipulation of the valve if desired. For example, if the intraluminal scaffold assembly is deployed in an internal jugular vein of the patient, an external pressure can be applied manually to the neck at a location proximate the implant site of the intraluminal scaffold to shift the elongated core member and thus engage the valve. This advantage is further enhanced when a conforming scaffold is used as depicted in FIG. 3. That is, application of an external force will shift the conforming scaffold to manipulate the valve.

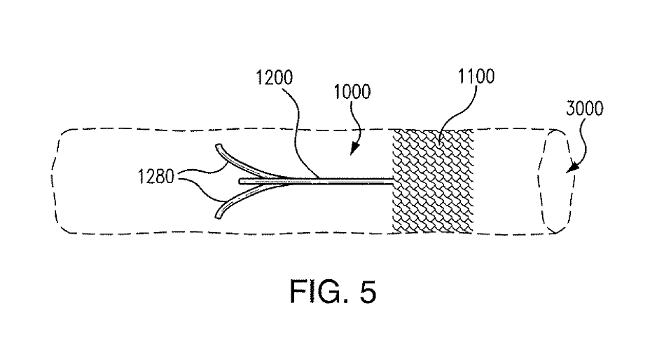

The intraluminal scaffold need not be implanted immediately proximate the valve to be manipulated, or in the same blood vessel in which the valve is located. For example, if the valve is located in an internal jugular vein, the intraluminal scaffold can be implanted in an external jugular vein 3040 on the same side of the patient's body, as illustrated in FIG. 4A, with the elongated core member extending into the internal jugular vein and across the valve. This configuration is particularly beneficial when using an intraluminal scaffold assembly having a weighted element coupled to the elongated core member. When the patient is in a standing posture, the weighted element can extend in a substantially vertical direction from the intraluminal scaffold, with the elongated core member substantially aligned with the axis of the internal jugular vein. Thus, minimal side load is placed on the internal jugular valve through which the elongated core member extends. This allows the valves to operate normally. As shown in FIG. 4B, when the patient is in a supine or prone position, the weighted element causes the elongated core member to deflect laterally within the internal jugular vein and engage the valve for increased blood flow.

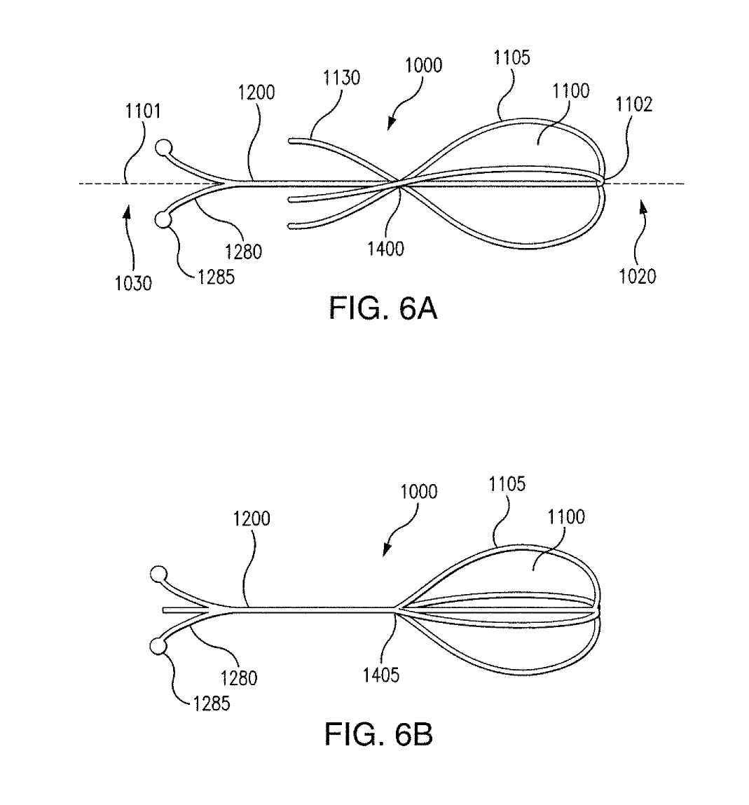

In some embodiments, the elongated core member of the intraluminal assembly described above can include a tail element extending laterally from the elongated core member. Referring, for example, to FIG. 5, the intraluminal scaffold assembly 1000 includes an intraluminal scaffold 1100. The intraluminal scaffold assembly 1000 also includes an elongated core member 1200 coupled to the scaffold 1100. The elongated core member 1200 includes at least one tail element 1280 extending laterally from the elongated core member 1200.

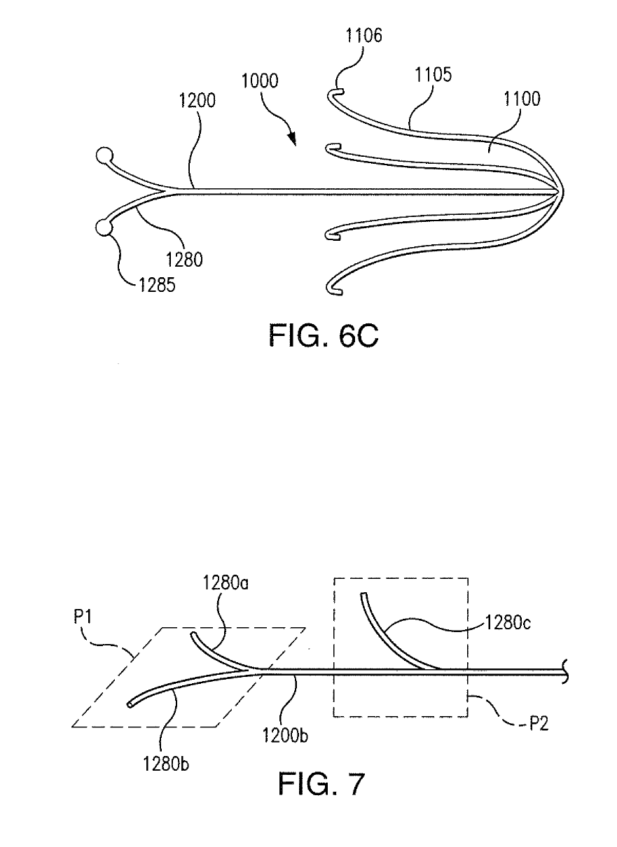

For purpose of illustration and not limitation, FIG. 6A shows a representative embodiment of intraluminal scaffold assembly 1000, which includes a conforming intraluminal scaffold 1100 as substantially described above in connection with FIG. 2, and the elongated core member including two tail elements 1280. It is appreciated that the elongated core member 1200 can be configured relative to the scaffold 1100 in manners other than shown in FIG. 6A. For example, the elongated core member can be coupled with the filaments at the juncture either alone or in addition to being coupled to the head portion. Alternatively, as depicted in FIG. 6B, the ends of the filaments of the intraluminal scaffold are joined together at location 1405, and the elongated core member can be coupled with the filaments at the same location 1405. As another alternative, as shown in FIG. 6C, the filaments of the scaffold can diverge as they extend longitudinally away from the head portion and take a splayed configuration at their free ends 1106. It is desirable that the free ends of the filaments (shown in FIGS. 6A and 6C), as well as the other portions of the filaments, are atraumatic to minimize injury to the lumen wall during the deployment of the scaffold assembly or while the scaffold assembly is implanted in the body lumen.

As previously noted, the elongated core member can take a variety of configurations and be made of a variety of materials. The tail elements 1280 can also take a variety of configurations. For example, as illustrated in FIGS. 5 and 6, the two tail elements each extend laterally from the elongated core member 1200. The two tail elements can extend in generally opposite lateral directions from each other, e.g., symmetrically with respect to the elongated core member, as shown in FIGS. 5 and 6, or the tail elements can both be generally directed to the same direction. Each of individual tail elements can also configured differently, e.g., curved or shaped as desired or needed, to adapt to the geometry of the body lumen, or to the geometry or other characteristics of the valve to be manipulated.

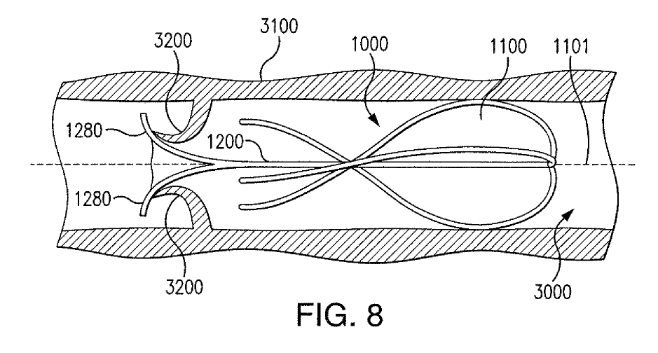

The tail elements can extend from a same location or different locations on the elongated core member. The location can be at an intermediate portion of the elongated core member (shown in FIGS. 5 and 6B), or an end of the elongated core member (shown in FIG. 6A). Further, the intraluminal scaffold assembly can include three or more tail elements each extending laterally from the elongated core member. The axial and angular distribution of the three or more tail elements can be selected based on the geometry of the body lumen, the valve to be manipulated, or any other desired objectives. For example, the three or more tail elements can extend from a same location on the elongated core member or from different locations along the elongated core member. As shown in FIG. 7 (the scaffold is not depicted), the intraluminal scaffold assembly includes three tail elements 1280a, 1280b, and 1280c, wherein two of the three tail elements, 1280a and 1280b, extend from a same location along the elongated core member, and the third tail element 1280c extends from a second location along the elongated core member which is relatively closer to the intraluminal scaffold (not shown). As shown, the tail elements 1280a, 1280b are on a plane P1 which is unparallel with the plane P2 which comprises tail element 1280c. The tail element 1280c can be referred to as a trigger, which can facilitate the tail elements 1280a and 1280b to adopt a certain biased configuration, e.g., so as to bend in a direction opposite the direction of the trigger divergence. Other configurations of the trigger and the remaining tail elements can be used as needed or desired.

When the intraluminal scaffold assembly includes a plurality of tail elements, the tail elements can have a same length or different lengths, and each constructed from a same or different material. The material for the tail elements can be, for example, a stainless steel, a shape memory alloy such as nickel-titanium, other metals or metal alloys such as cobalt-chromium, or polymers such as nylon, PTFE, or composites as appropriate. Additionally, each tail element can have an atraumatic end 1285 (such as shown in FIGS. 6A and 6B) to avoid or minimize injury to the vessel wall of the body lumen or the valve of the body lumen. The atraumatic end can have a variety of shapes, and can be made of the same or different material as the tail element.

The tail elements can be formed as an extension of reduced cross-section of the elongated core member, or can be formed as a separate element and attached to the elongated core member using a conventional process such as welding. It is also contemplated that one or more of the tail elements to be formed as extensions of the filaments of the intraluminal scaffold. For example, for the embodiment of the intraluminal scaffold assembly as depicted in FIG. 6B, the filaments 1105 can extend through the elongated core member (e.g., by braiding or otherwise bundling a portion of the filaments), and the tail elements can be formed as part of the filaments that extend from the elongated core member. Hence, the tail element can be made of the same or different material as the elongated core element or the filaments of the intraluminal scaffold. Further, each tail elements can include branched configurations, i.e., include “finer tails.”

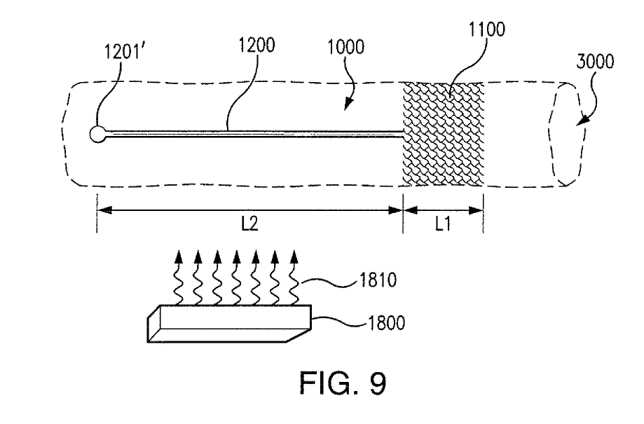

As embodied herein and as schematically illustrated in FIG. 8, the tail elements of the intraluminal scaffold assembly can be used to manipulate a valve in the body lumen of a patient. For example, the intraluminal scaffold assembly 1000 can be implanted within a body lumen, e.g., a blood vessel 3000 having a wall 3100 with the tail elements 1280 disposed across a valve 3200 of the blood vessel 3000.

With the tail elements extending laterally from the elongated core member, the tail elements can exert lateral forces to engage or prop open the valve leaflets. Further, incidental movement by the patient can result in movement of the tail elements and thus changing the opening status of the leaflets. As there are a variety of possible configurations of tail elements, it is not necessary for all tail elements engage the valve at the same time. For example, if the intraluminal scaffold assembly include a series of tail elements along the elongated core member, it is contemplated that only one or a few closely spaced tail elements to engage the valve at one time. Thus, when the intraluminal scaffold assembly shifts position as a result of compression or dilation of the blood vessel or incidental move of the patient, different tail elements can engage the valve. The blood vessel can be a vein, e.g., an internal jugular vein or external jugular vein.

It is noted that FIG. 8 depicts the intraluminal scaffold implanted upstream of the valve 3200, with the elongated core member extending downstream. By contrast, the intraluminal scaffold can be implanted downstream with the elongated core member extending upstream, or the valve can be malformed so as to be inverted as shaped different than as shown.

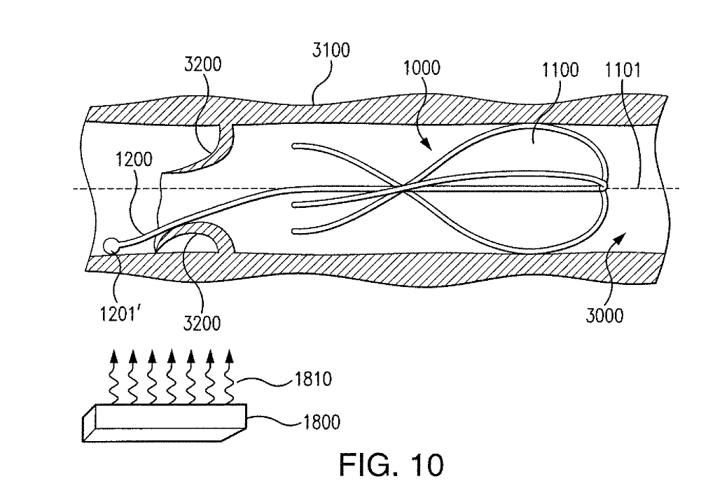

According to another aspect of the disclosed subject matter, the intraluminal scaffold assembly can further include an active element coupled with the elongated core member at a location spaced from the intraluminal scaffold, wherein the active element is externally actuatable to deflect the elongated core member. Referring, for example, to FIG. 9, the intraluminal scaffold assembly 1000 includes an intraluminal scaffold 1100 having a longitudinal length L1. The intraluminal scaffold assembly 1000 also includes an elongated core member 1200 coupled to the scaffold 1100. The elongated core member 1200 includes a portion having a length L2 extending beyond the length L1 of the scaffold 1100 to traverse a valve in the body lumen. An active element 1201′ is coupled to the elongated core member 1200 and spaced from the intraluminal scaffold 1100, wherein the active element can be actuated by an external source to manipulate the valve, e.g., to temporarily open the valve using the elongated core member.

For purpose of illustration and not limitation and with renewed reference to FIG. 2, an intraluminal scaffold assembly 1100 is shown, which includes a conforming scaffold 1100 previously described in connection with FIG. 2, an elongated core member 1200 coupled with the scaffold 1100, and further an active element 1201′ coupled to the elongated core member.

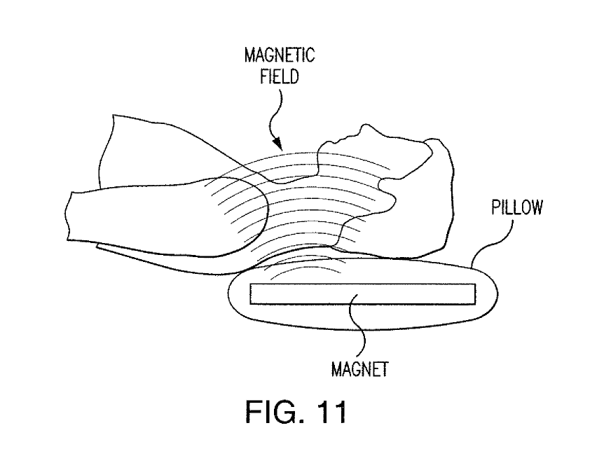

As embodied herein and as schematically illustrated in FIG. 10, the elongated core member of the intraluminal scaffold assembly can be used to manipulate a valve in the body lumen of a patient. For example, the intraluminal scaffold assembly 1000 can be implanted within a body lumen, e.g., a blood vessel 3000 having a wall 3100 with the elongated core member 1200 disposed across a valve 3200 of the blood vessel 3000. The active element 1201′ can provide enhanced and more controlled manipulation by actuation using an external field or the like. It is noted that FIG. 10 depicts the intraluminal scaffold implanted upstream of the valve 3200, with the elongated core member extending downstream. By contrast, the intraluminal scaffold can be implanted downstream with the elongated core member extending upstream, or the valve can be malformed so as to be inverted as shaped different than as shown.

The active element 1201′ is configured to respond to an externally applied field to cause the elongated core member to deflect. As shown in FIGS. 9 and 10, the active element 1201′ can have an enlarged profile relative to the rest of the elongated core member. Additionally, the active element can be provided with a mass suitable to cause or assist in applying a torque to the elongated core member. For generating a greater torque to cause a deflection of the elongated core member, the active element can be placed at a free end the elongated core member, as shown in FIGS. 9 and 10, although the active element can be coupled to the elongated core member at intermediate locations if preferred or needed. The active element can be magnetically active, e.g., the active element can be or include a magnet, or include a material that can be magnetically attracted or repelled by a magnetic field. The active element can further include a coating layer such as biocompatible polymer.

The intraluminal scaffold assembly can further include a source of external field to actuate the active element. As illustrated in FIG. 9, a source 1800 provides an external field 1810 to actuate the active element 1201′. For example, if the active element is magnetically active, the source of external field can include one or more magnets, such as permanent magnets or electromagnets. The magnet(s) or other source of external field can be disposed within a housing structure configured for ease of use. For example and not limitation, such a structure can be a pillow for use when the patient is in a prone or supine position, such as when lying down or sleeping. Alternatively, the housing structure can be an article to be worn by the patient, such as a neck brace or collar or the like. The source of external field can generate an adjustable field strength, orientation, or a combination thereof, and can include a controller or controllers for the desired adjustment. For example, an adjustable magnetic field can be generated by a plurality of selectively adjustable magnets distributed at different locations (including depths and/or lateral displacements) in a housing. Each magnet can be activated individually or in combination to create and apply an external field with different orientation and/or strength from the housing. Additionally, the strength of each magnet can be adjusted to increase or decrease as desired. Thus, the strength, orientation, or a combination thereof of the applied field can be controlled, e.g., by activating different number of magnets and/or different magnets distributed at different locations. Alternatively, the strength and/or orientation of the external field can be controlled by changing the position of the magnets in the housing structure, e.g., via rotation, translation or other techniques. Additionally, a controller can be provided to control the desired activation and/or adjustment of the external source. The controller can further provide a varying strength and/or orientation according to a preset schedule as desired by a user. For example, if a constant blood flow is desired, a steady magnetic field can be used to manipulate the valve in an open condition. If a periodic opening of a venous valve is desired, the source of the magnetic field can be set to periodically activate and deactivate the magnetic field.

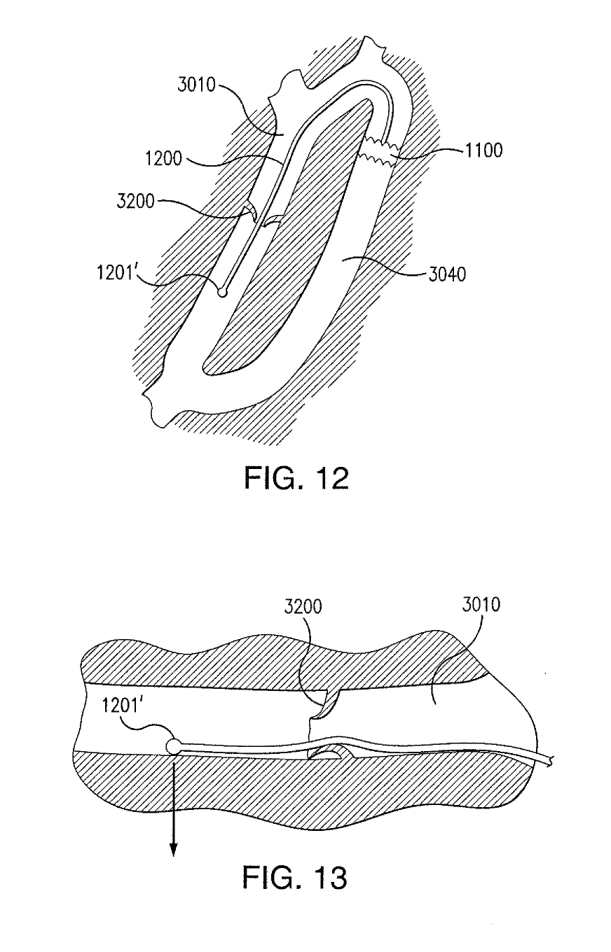

As embodied herein for illustration and not limitation, the external field is a magnetic field. As shown in FIG. 11, when the external field is activated, the field will propagate through the patient anatomy. The external field can either repel or attract the active element of the intraluminal scaffold assembly implanted in the patient's internal jugular vein. Although not shown, it is appreciated that the external field can be adjustable, e.g., to vary strength or orientation, and/or can be controlled or programmed as desired.

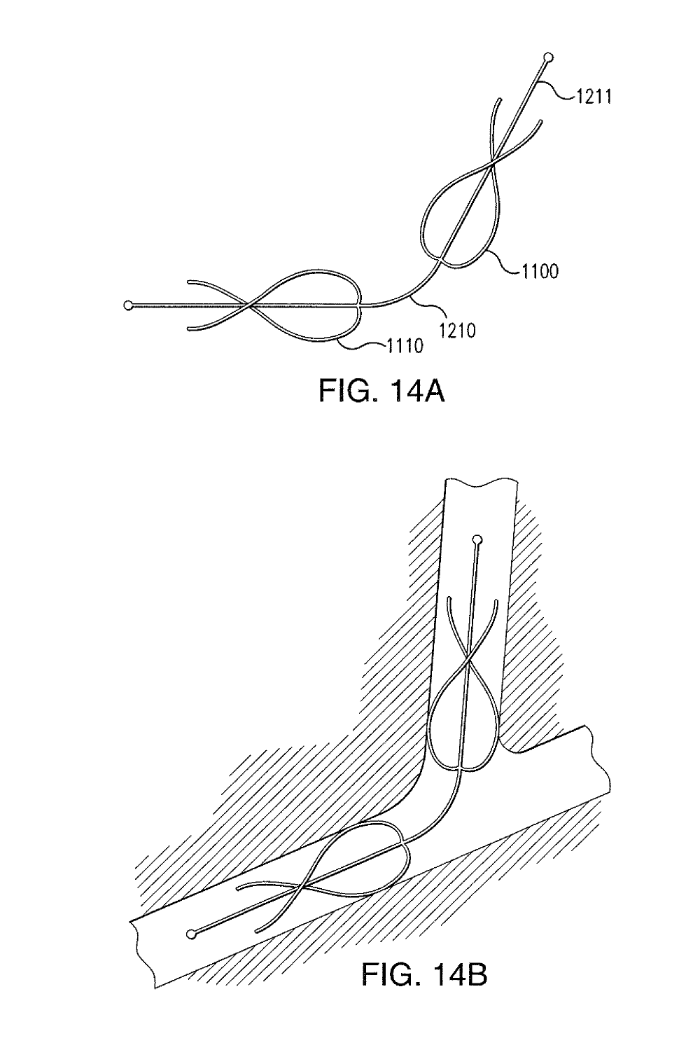

In conjunction with the intraluminal scaffold assembly described above that includes an active element, a method of manipulating a valve of a body lumen in a patient is provided. The method includes deploying an intraluminal scaffold assembly in a body lumen of a patient as generally described above, and applying an external field to activate the active element to deflect the elongated core member. For purpose of illustration and not limitation, an alternative embodiment of intraluminal scaffold system as implanted is shown in FIG. 12. The body lumen can be a blood vessel, e.g., an internal jugular vein as shown. The intraluminal scaffold can be implanted in the same body lumen, e.g., the internal jugular vein, either upstream or downstream proximate the valve to be manipulated. Alternatively, the intraluminal scaffold can be implanted in a different blood vessel. For example, as shown in FIG. 12, the intraluminal scaffold is implanted in the external jugular vein 3040, whereas the valve to be manipulated, 3200, is located in the internal jugular vein 3010. In this manner, the elongated core member 1200 extends from the external jugular vein into the internal jugular vein. This configuration can improve the positional stability of the intraluminal scaffold assembly as implanted because the bend of the elongated core element at the crossing of the internal jugular vein and the external jugular vein can provide extra support for the weight of the section of the elongated core member in the internal jugular vein, including the active element when the patient is in a standing posture. It is appreciated that to generate a greater torque and reduce the mass or the volume of the active element, the active element can be disposed on the side of the valve opposite the location of the intraluminal scaffold, as shown in FIG. 12.

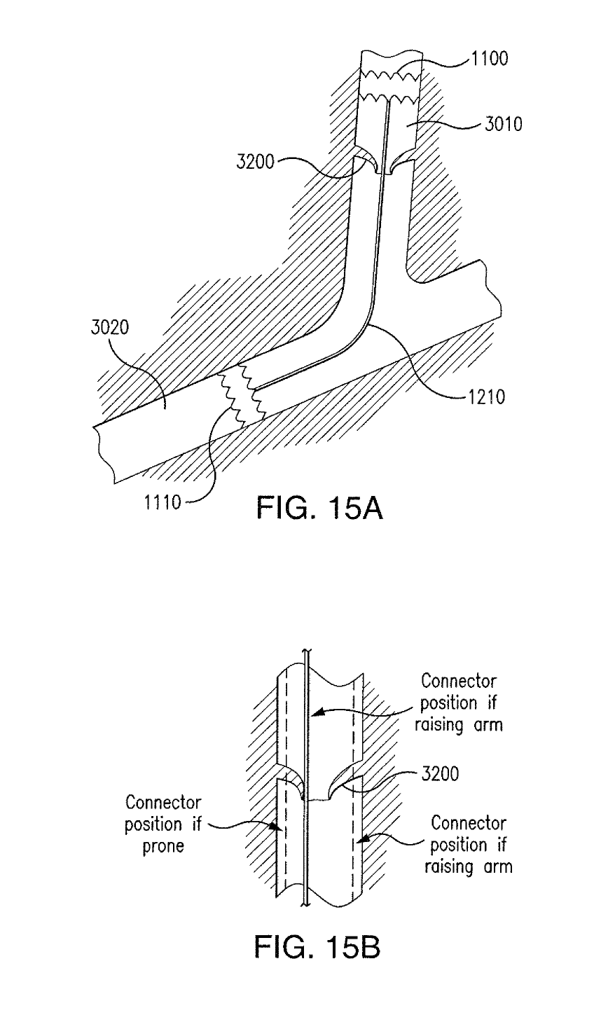

FIG. 13 illustrates the effect of the magnetic field on the intraluminal scaffold assembly as implanted. When the magnetic field is applied, the active element 1201′ is actuated through magnetic attraction or repulsion to produce a corresponding effect on the disposition of the elongated core member. For example, if the magnetic field pulls the active element of the intraluminal scaffold, the active element can cause the elongated core member to deflect toward the source of the external field. In turn, the elongated core member traversing the valve can contact and urge open a valve leaflet, thereby allowing greater blood flow across the valve. As discussed above, the magnetic field can be adjusted by controlling its strength, orientation, timing, or a combination thereof, e.g., by selectively activating/deactivating a plurality of magnets embedded in a housing structure, e.g., a pillow.

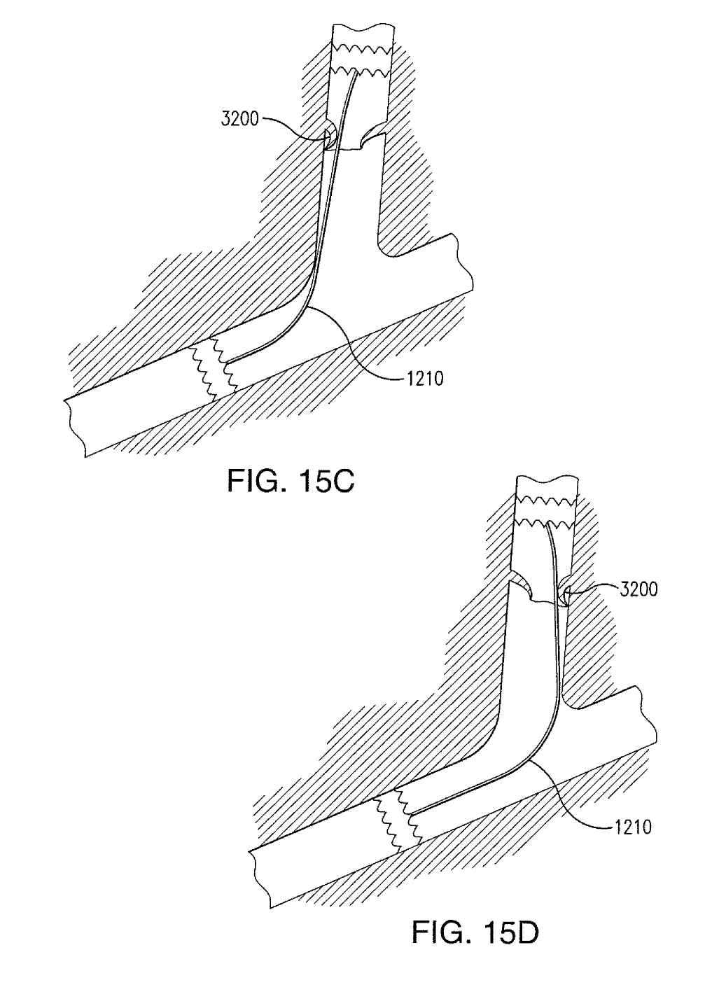

According to another aspect of the disclosed subject matter, the intraluminal scaffold assembly can further include a second intraluminal scaffold coupled with the elongated core member and spaced from the first intraluminal scaffold. As illustrated for purpose of illustration and not limitation in FIG. 14A, a first intraluminal scaffold 1100 is connected with a second intraluminal scaffold 1110 via an elongated core member 1200. As embodied herein, the elongated core member 1200 in FIG. 14A can include several portions: (1) a portion 1210 disposed between the two scaffolds 1100 and 1110, referred herein to as the connector portion 1210, (2) a portion coextensive with the length of the length L1 of each scaffold, respectively, and (3) if desired, a portion extending beyond the outward end of each scaffold, respectively, referred to herein as the extended portion 1211. The intraluminal scaffold assembly can be positioned in a body lumen, for example, a blood vessel(s), with either the connector portion or the extended portion of the elongated core member traversing a valve. Additionally, the tip of at least one of the extended portion of the elongated core member 1200 can be shaped or configured to be used as an engaging element with a delivery system. As such, the different portions of the elongated core member can have different characteristics or properties, and can be constructed of different materials.

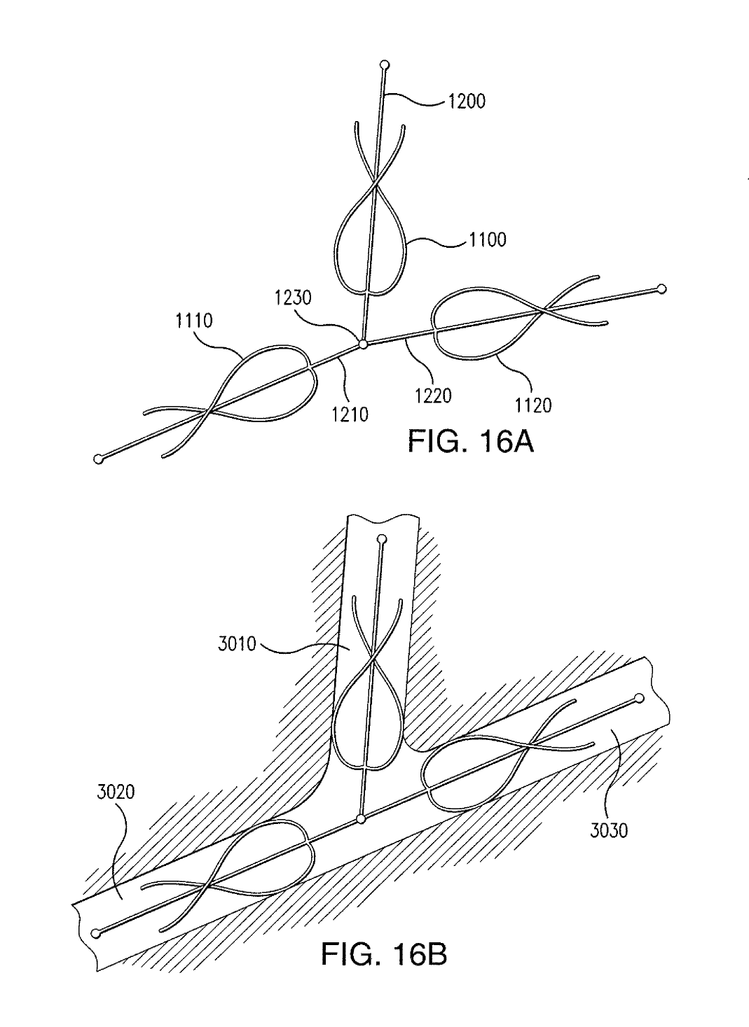

It is appreciated that the intraluminal scaffold assembly as depicted in FIG. 15A can also have improved positional stability when implanted in the vasculature of a patient. That is, the use of two scaffolds provide two possible “points of contact” for the assembly within the lumen. The improved positional stability can prevent or inhibit the assembly from shifting downstream or otherwise dislodging from position. Additionally, the elongated core member can have a portion biased in a non-linear shape to further enhance positional stability of the assembly within or near a vessel bifurcation or bend. Referring to FIG. 14A, the curved connector 1210 creates an angular offset between the axis of the first intraluminal scaffold and the second intraluminal scaffold. This connector portion can have a radius, a discrete angulation, or a combination of the two. The biased portion of the elongated core member can be configured to have suitable flexibility to facilitate delivery within the vasculature and to facilitate conformability once placed inside of the vasculature. It is appreciated that the flexibility of the connector is a function of both dimensions and material, which can be balanced to achieve the desired connector characteristics.

FIG. 14B illustrates placement of an intraluminal scaffold assembly having a portion of the elongated core member between the first intraluminal scaffold 1100 and the second intraluminal scaffold 1110 biased in a non-linear shape. In this example, the intraluminal scaffold assembly is positioned within a vessel bifurcation, e.g., where the internal jugular vein and subclavian veins join with the brachiocephalic vein. The bend of the connector allows for the intraluminal scaffolds to be located within the adjacent veins in a natural position. That is, there is minor bending moments applied to the connector because its shape approximates the bend of the venous anatomy.

As embodied herein, a connector portion of the elongated core member between two connected scaffolds, such as those depicted in FIGS. 14A and 14B, can be used to manipulate a valve. For example, and with reference to FIGS. 15A-15D, which depict two scaffolds 1100 and 1110 that are connected via a connector portion 1210, and implanted proximate a bifurcation point of the vasculature. Although FIGS. 15A and 15C-15D depict the intraluminal scaffold assembly with supporting scaffolds, the description is also generally applicable to an intraluminal scaffold assembly wherein one or both of the intraluminal scaffolds are a conforming scaffold.

As illustrated in FIG. 15A, a first intraluminal scaffold 1100 can be implanted in a proximal portion of an internal jugular vein 3010. A second intraluminal scaffold 1110 can be placed distal to the first intraluminal scaffold 1100. For example, the second intraluminal scaffold can be located in a distal or downstream portion of the internal jugular vein (not shown), or in the brachiocephalic vein 3020 as shown. The elongated core member passes through at least one venous valve 3200 located between the two intraluminal scaffolds.

Referring to FIG. 15B, it is appreciated that the location of the elongated core member within the venous lumen can vary depending upon the posture of the patient. For example, while the patient is in a first position, the distance between the intraluminal scaffolds as a result of the orientation of the veins can decrease, thereby producing a slackness in the elongated core member and allowing the elongated core member to move toward the center of the vein and the venous valve. Alternatively, the location of the connector portion 1210 may shift laterally when the distance between the two scaffolds 1100 and 1110 increases or decreases, such as when the patient shifts position or raises his or her arm. It will be appreciated that specific changes in distance between the two scaffolds may correspond to different postures than those described above.

As a further example, the elongated core member between the two scaffolds as shown in FIG. 15A can be biased in a non-linear shape, such that when implanted, the elongated core member can apply a lateral force to keep the valve 3200 open. This is illustrated in FIGS. 15C and 15D. Depending on how the bias is set and the difference between the unconstrained shape of the elongated core member and the vessel geometry, the elongated core member can apply different lateral forces, e.g., a side force on one side of the valve as shown in FIG. 15C, or a side force on the other side of the valve as shown in FIG. 15D. In this manner, the valve can be urged to stay open at all times or independent of the patient's posture or movement. While implanted, the intraluminal scaffold assembly thus can maintain the venous valve in an open configuration if desired. Also, over time the intermittent influence on the venous valves can lead to a tendency of the valves to self-open, such as when a patient lies down. This can occur, for example, as a result of the venous valves gaining a shape set or increased tissue stiffness in certain areas.

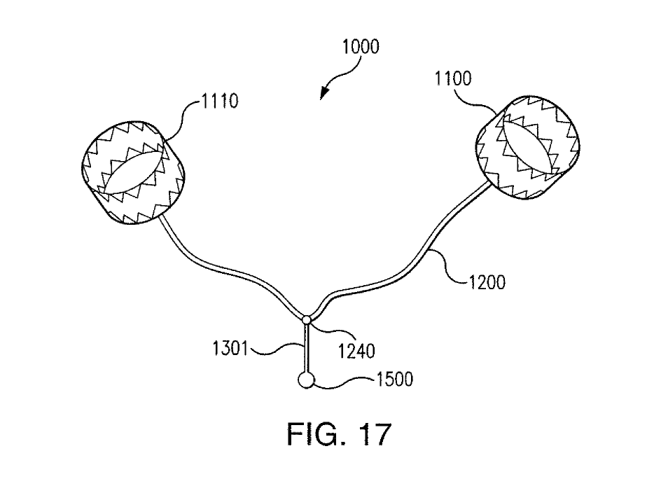

According to a further aspect of the disclosed subject matter, the intraluminal scaffold assembly can further comprise a second elongated core member extending from the first elongated core member at a connection location between the first intraluminal scaffold and the second intraluminal scaffold, and a third intraluminal scaffold coupled with the second elongated core member. As an illustration, FIG. 16A shows an intraluminal scaffold assembly including the first intraluminal scaffold 1100, the second intraluminal scaffold 1110, and the third intraluminal scaffold 1120. A second elongated core member 1220 extends from the connection location 1230 between the first elongated core member 1200 and the second elongated core member 1220. The third intraluminal scaffold 1120 is coupled to the second elongated core member. As shown, each individual scaffold can be a conforming scaffold as shown and previously described in connection with FIG. 2, or can be other positional elements such as supporting scaffolds (e.g., stents), anchors, or the like. As previously described, various portions of the elongated core members 1200 and 1220 can be used for traversing and manipulating a valve.

A hinge can be defined between the first elongated core member and the second elongated core member at the connection location 1230 as shown in FIG. 16A. The hinge can have a variety of constructions. For example, the hinge can be a living hinge or an area of relative weakness, or the hinge can include swivel-type hinge in which the hinge itself is a ring through which the elongated core members are attached. The hinge allows each of the elongated core members to move about the hinge. In an alternative embodiment, the hinge can be a u-bolt construction in which the one of the connector portion has a u-shape tip and the other connector portions are pinned to the u-shape. The hinge can also be a simple flexible bond formed between the connector portions of each scaffold. For example, an elastomeric adhesive can be used to form a node that permits some flexibility and movement between the connector portions. In this manner, a flexible and/or elastic articulation point can be created. In the above embodiments, the first elongated core member can be considered as including the hinge.

FIG. 16B illustrates the intraluminal scaffold assembly illustrated in FIG. 16A as deployed within the vasculature of a patient. As embodied herein, the three intraluminal scaffolds are deployed in an internal jugular vein 3010, a brachiocephalic vein 3020, and a subclavian vein 3030, respectively, which are joined at a bifurcation point. The hinge can be positioned near the carina of the bifurcation to create minimal resistance to blood flow through this portion of the vasculature. The elongated core members can be constructed and function in the manners previously described to manipulate the valve as desired.

In accordance with another aspect of the disclosed matter, an intraluminal scaffold assembly is provided that includes a first intraluminal scaffold; a second intraluminal scaffold, and an elongated core member coupled with each of the first intraluminal scaffold and the second intraluminal scaffold, the elongated core member including a connecting portion between the first intraluminal scaffold and the second intraluminal scaffold, the connecting portion having a length sufficient to traverse a valve in a body lumen of a patient, and a weighted node coupled to the elongated core member at a location between the first intraluminal scaffold and the second intraluminal scaffold. Further, a method of deploying such an intraluminal scaffold assembly is provided. The method generally includes implanting the first intraluminal scaffold in a first target site, implanting the second intraluminal scaffold at a second target site, and disposing the elongated core member to cross a valve of the body lumen. The method will be described in conjunction with the intraluminal scaffold assembly and system below.

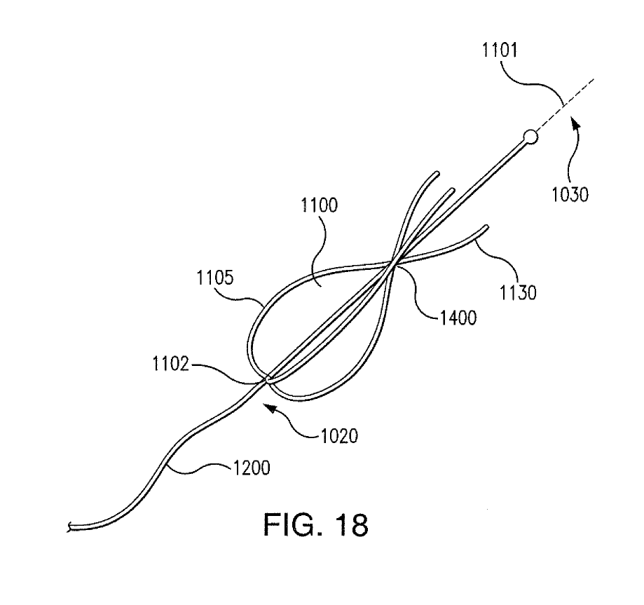

Referring to FIG. 17, the intraluminal scaffold assembly 1000 includes a first intraluminal scaffold 1100 and a second intraluminal scaffold 1110, and an elongated core member 1200 coupled to each of the first intraluminal scaffold 1100 and the second intraluminal scaffold 1110. The intraluminal scaffold assembly 1000 also includes a weighted node 1500 coupled to the elongated core member 1200 at a location 1240 between the first intraluminal scaffold 1100 and the second intraluminal scaffold 1110. The length of the elongated core member between the first intraluminal scaffold 1100 and the second intraluminal scaffold 1110 is sufficient to traverse at least one valve in a body lumen of a patient, depending on the intended application and the deployment site of the intraluminal scaffold assembly.

The first intraluminal scaffold 1100 and the second intraluminal scaffold 1110 can both have an expanded profile to provide positional stability of the intraluminal scaffold assembly in the body lumen as deployed. A variety of suitable types of intraluminal scaffolds can be used. For example, in certain circumstances, a supporting scaffold, such as a stent, spiral, anchor, or the like can be used as the intraluminal scaffold in the intraluminal scaffold assembly 1000. Alternatively, in certain circumstances, such as when anchoring into the lumen wall is not necessary or desired, the intraluminal scaffold can be a conforming scaffold as described above.

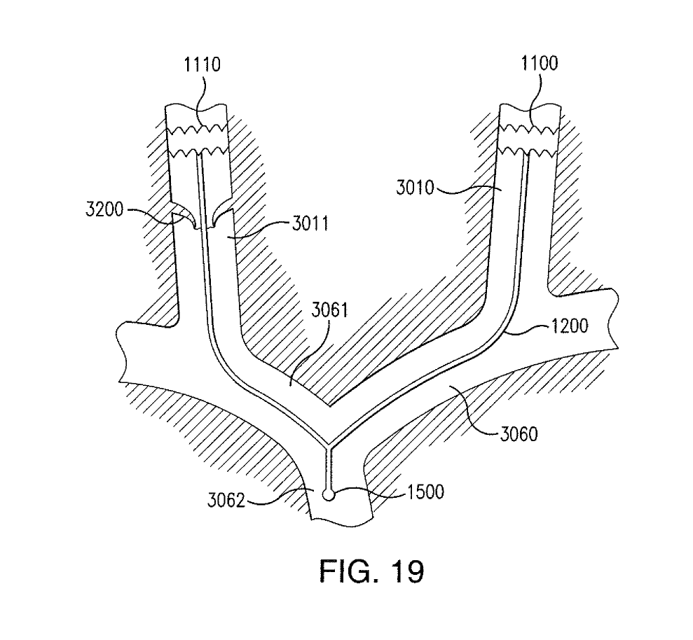

For purpose of illustration and not limitation, FIG. 18 shows a representative embodiment of a portion of the intraluminal assembly of FIG. 17, when one or both of the supporting scaffold are replaced with a conforming scaffold, respectively. The conforming scaffold as depicted herein has been substantially described in connection with FIG. 2, above, As shown in FIG. 18, the elongated core member 1200 is coupled to the intraluminal scaffold 1100 at the head portion, extending in one direction through the juncture 1400 to define a tip and in the other direction away from the juncture 1400 to define a connecting portion. It is appreciated, however, that the elongated core member 1200 can be configured relative to the intraluminal scaffold 1100 in other manners. For example, the elongated core member can be coupled with the filaments at the juncture either alone or in addition to being coupled to the head portion.

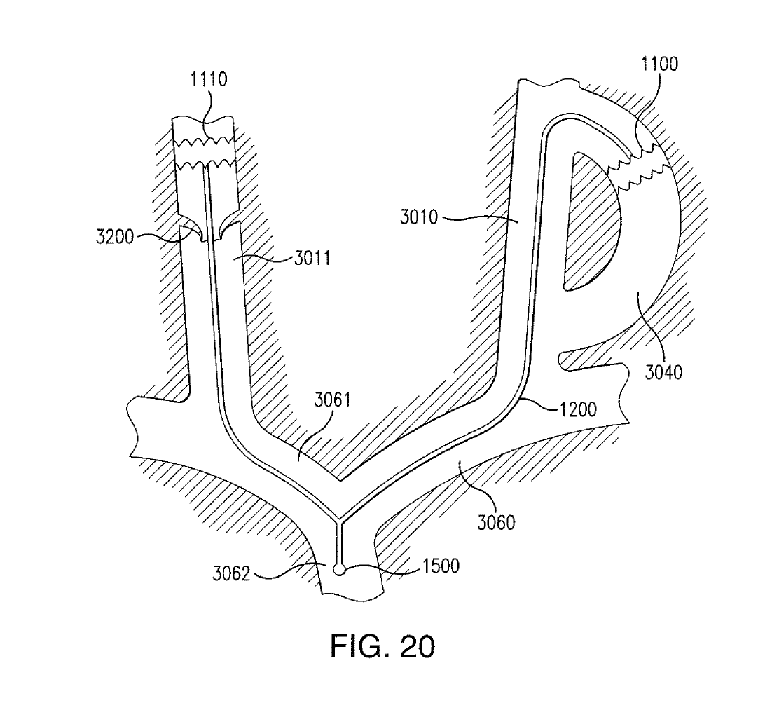

The mass of the weighted node 1500 can be selected to be sufficient to bend the elongated core member by gravitational force from its tension-free position. For example, if the intraluminal scaffold assembly is deployed in the vasculature of a patient as shown in FIG. 19, which will be further described below, the flexibility of the elongated core member and the mass of the weighted node can be selected such that the elongated core member generally does not interfere the normal operation of the valve when the patient is in a standing posture, but urges the valve into an open configuration when the patient is in a supine, prone, or otherwise horizontal position. The weighted node 1500 can be coupled to the elongated core member directly or by a depicted connector, such as wire segment 1301, as shown in FIG. 17. The coupling can be achieved by welding, bonding or any other means as appropriate. In addition, the dimension and shape of the node can be selected to allow the weighted node to be deployed into the desired location of the patient's vasculature and move relatively freely within the deployed location.

The intraluminal scaffold assembly as described above can be deployed as follows: implanting the first intraluminal scaffold at a first target site, implanting the second intraluminal scaffold at a second target site, and disposing the elongated core member to cross a valve in at least one of the left internal jugular vein or the right internal jugular vein of the patient. This method is further illustrated in reference to FIG. 19 below, which depicts the intraluminal scaffold assembly as deployed in accordance with the above method. As embodied herein, for purpose of illustration and limitation, the assembly is depicted with supporting scaffolds. However, one or both scaffolds can be replaced with conforming scaffolds as desired or needed. As shown, the first intraluminal scaffold and the second intraluminal scaffold are implanted in the left and right internal jugular vein 3010 and 3011, respectively. The elongated core member extends between the left internal jugular vein 3010 and the right jugular vein 3011 by crossing the left brachiocephalic vein 3060 and the right brachiocephalic vein and 3061, respectively. The two scaffolds are positioned such that the elongated core member 1200 traverses at least one valve of either the left internal jugular vein and the right internal jugular vein. As depicted in FIG. 19, a valve 3200 in the right internal jugular vein is shown for purpose of illustration, but it is appreciated that more than one valve either in the left internal jugular vein or the right internal jugular vein can be traversed by the elongated core member.

The deployed configuration of the intraluminal scaffold assembly illustrated herein allows the valve to be manipulated by a change in posture of the patient, such as a neck turn or raising an arm, which can cause the elongated core member to move axially along the direction of the internal jugular vein(s) and/or laterally against the wall of the internal jugular vein(s) to temporarily open a valve therein. The bilateral span of the intraluminal scaffolds positions enhance actuation of the weighted node by which changes in posture can be transferred over an extended length of the elongated core member. Particularly, the torque applied to the elongated core member increases by the bilateral span of the assembly for manipulation of the valve traversed by the elongated core member.

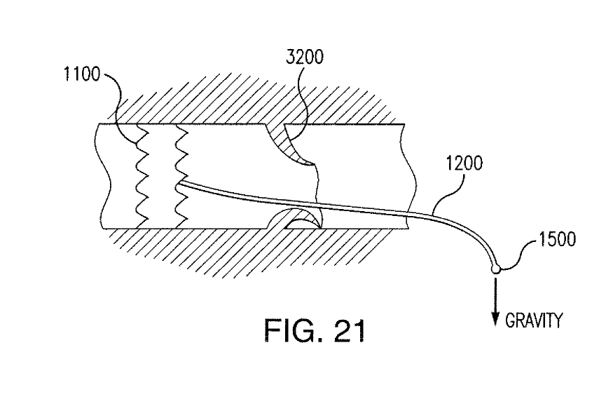

Alternatively, the first intraluminal scaffold and/or the second intraluminal scaffold can also be implanted in a body lumen other than an internal jugular vein. For example, as illustrated in FIG. 20, the first intraluminal scaffold can be implanted in the left external jugular vein 3040 rather than the left internal jugular vein. The elongated core member extends from the first intraluminal scaffold 1100 through the left external jugular vein, enters into the left internal jugular vein 3010, and then extends to the right internal jugular vein, as depicted in FIG. 19. Additionally or alternatively, the second intraluminal scaffold can also be implanted in a vessel other than the right internal jugular vein, such as in the right external jugular vein, and either scaffold can be a conforming scaffold if preferred or needed.

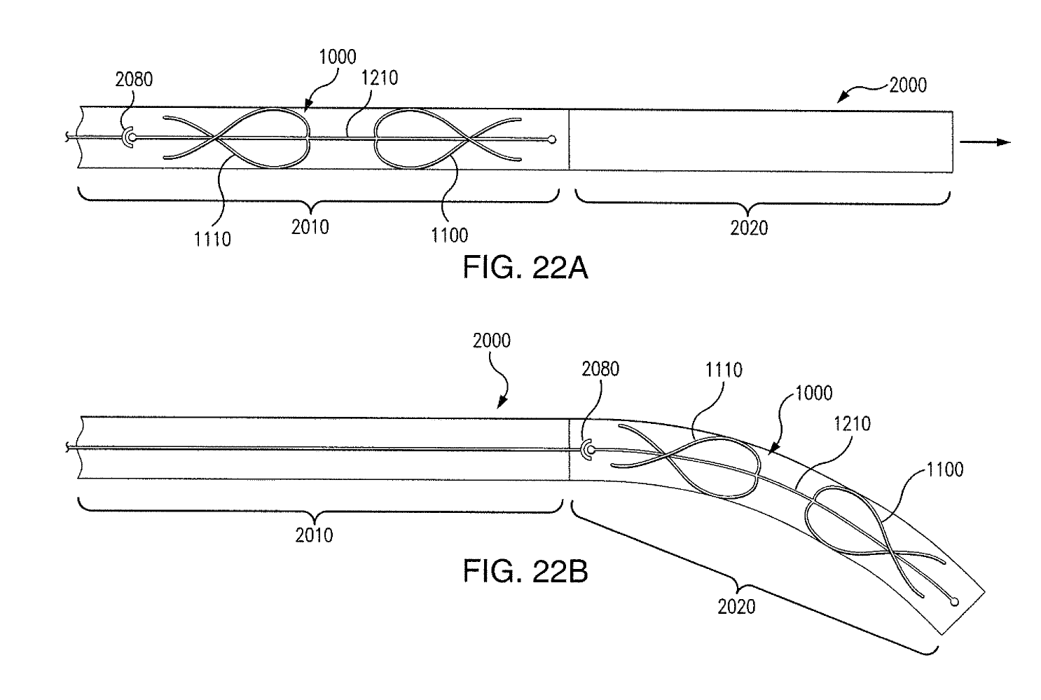

When the intraluminal scaffold assembly includes a weighted node 1500 as depicted, additional mode of controlling the internal jugular valves can become available. As shown in FIG. 19, the weighted node 1500 can be deployed to be positioned proximate the upper end of superior vena cava where the left and right brachiocephalic veins meet. It is appreciated that the schematic drawings shown in FIGS. 19 and 20 show the deployed scaffold assembly when the patient is in an upright position. At this posture, the weighted node causes a tension in the elongated core member. However, as the elongated core member extends along the left internal jugular vein and right internal jugular vein substantially vertically, such a tension align the elongated core member along the internal jugular veins and but would not urge open an internal jugular valve. Thus, minimal side load is placed on the valve through which the elongated core member extends. This allows the valve to operate normally. As shown in FIG. 21, when the patient is in a supine or prone position, the weighted node 1500 causes the elongated core member 1200 to deflect laterally within the internal jugular vein and engage the valve for increased blood flow.

In accordance with another aspect of the disclosed subject matter, an intraluminal scaffold system is provided. The system includes a delivery system, e.g., a delivery catheter, which can be of similar construction and operation as contemplated for delivering self-expanding stents or the like. See, for example, U.S. Pat. No. 7,799,065 to Pappas, the contents of which are incorporated by reference in its entirety. For the example, the delivery catheter can include an inner member having a distal end portion and an outer sheath generally surrounding and movable relative to the inner member. The outer sheath defines a catheter lumen and has a first position to cover the distal end portion of the inner member and a second position to expose the distal end portion of the inner member. The delivery catheter can have a distal portion including two sections of different longitudinal stiffness, where the more distal section can bend or be steered by an intraluminal scaffold assembly disposed therein, as further described below. The intraluminal scaffold system also includes any of the various embodiments of intraluminal scaffold assembly as previously described disposed at the distal end portion of the inner member to releasably engage the distal end of the inner member of the delivery system.

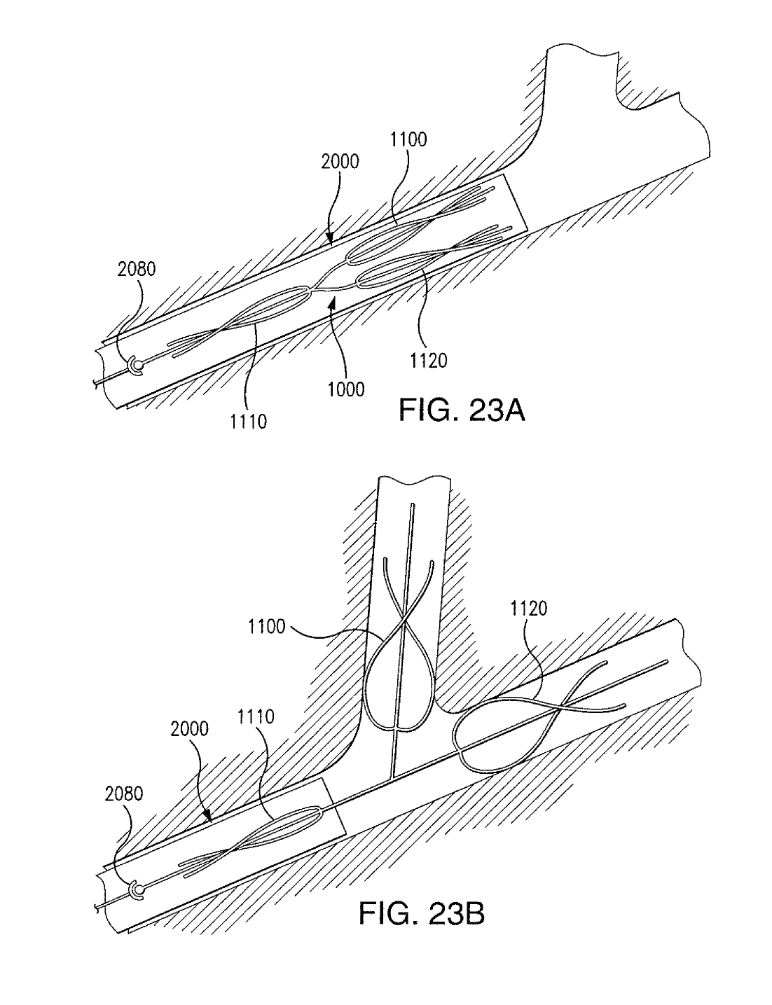

The outer sheath of the delivery catheter can be made of any suitable material as known in the art, including single layer or multi-layer construction, and is sized and configured to constrain the intraluminal scaffold assembly in a low profile condition. The elongated core member of the intraluminal scaffold assembly can include a tip to releasably engage the inner member of the catheter. Generally, the inner member has a distal end configured to engage or mate with the elongated core member of the intraluminal scaffold assembly in a stable manner. For example, the inner member can have a cup geometry, as illustrated in FIGS. 22A and 22B, which will be further described below. Alternatively, the distal portion of the inner member can have a tube with a back support geometry, or any other geometry to engage the tip of the elongated core member of the intraluminal scaffold assembly. As an alternative, the inner member can engage another portion of the intraluminal scaffold assembly, such as the juncture or the free end portion of one or more filaments of a conforming scaffold. The inner member is generally configured for longitudinal strength but axial flexibility. For example, the inner member can be constructed from a metallic wire that extends the length of the catheter. The outer sheath is movable relative to the inner member to expose the intraluminal scaffold assembly at the distal portion of the inner member. Actuation can occur by manually pushing the inner member, or by retracting the outer sheath by conventional means of actuation such as the rotation of a knob or gear that engages the pusher wire, as known in the art of stent delivery. Various other actuation mechanisms and catheter features consistent with the disclosed subject matter can be provided. For example, the delivery catheter can be either configured for over the wire (OTW) or rapid exchange (RX) guidewire deployment.

in operation, the intraluminal scaffold system can be used for deployment of the intraluminal scaffold assembly. Accordingly, a method of delivery an intraluminal scaffold assembly includes providing an intraluminal scaffold system as described previously; positioning the delivery system with the distal end portion disposed in a body lumen of a patient; and deploying the intraluminal scaffold assembly by moving the outer sheath to the second position relative to the inner member to implant at least the first intraluminal scaffold at a first target site with the elongated core member disposed across a valve of the body lumen. If the intraluminal scaffold assembly includes additional scaffolds, the method can be modified to further implant the second scaffold at a second site, and/or the third scaffold at a third site, as described further below.

In the method above, it is appreciated that implanting the intraluminal scaffold and disposing the elongated core member across the valve can be accomplished in one integral step. For example, the intraluminal scaffold assembly can be first positioned at the distal portion of the delivery catheter and within the outer sheath in a configuration similar to its intended deployed configuration. The distal end of the catheter can then be advanced through a body lumen, such as a blood vessel(s), of the patient and across the valve to be manipulated. The intraluminal scaffold assembly is then deployed into the body lumen by advancement of an inner pusher of the catheter which releasably engages the scaffold, or by retraction of the outer sheath of the catheter. Upon the deployment of the intraluminal scaffold assembly in the desired configuration, the scaffold is implanted at the target site with the elongated core member traversing the valve. In the above procedure, either the intraluminal scaffold or the valve traversing portion of the elongated core member can be positioned more distally in the catheter, thereby allowing the placement the intraluminal scaffold either upstream or downstream of the valve.

Alternatively, the intraluminal scaffold and the elongated core member can be deployed sequentially. For example, the intraluminal scaffold can be implanted at a target site first, and then elongated core member, which is previously held in a compact position, can be extended by a pulling mechanism of the catheter, such that the elongated core member is disposed across the valve.

In further embodiments, the intraluminal scaffold assembly can be assembled at the site of use. For example, the intraluminal scaffold can be implanted separately first, and then the elongated core member can be attached and/or coupled to the implanted intraluminal scaffold to traverse the valve of interest.

Other variations of the procedure for carrying out the method can be easily devised by those ordinarily skilled in the art in view of the description above.

In the above methods, the body lumen can be the vasculature of a patient, e.g., a blood vessel or blood vessels, such as a vein or veins as described above, and the valve can be a venous valve, e.g., an internal jugular venous valve. The first intraluminal scaffold can be implanted in the same blood vessel which houses the valve. For example, if the valve is an internal jugular valve, the first intraluminal scaffold can be implanted in the internal jugular vein, e.g., either upstream or downstream of the valve. Alternatively, the first intraluminal scaffold can be implanted in a different blood vessel. For example, as illustrated above in connection with FIGS. 4A and 4B, the first intraluminal scaffold can be implanted in an external jugular vein with the elongated core member disposed to cross a valve in the neighboring internal jugular vein.

In the above methods, if the elongated core member includes a weighted element, as illustrated in and described in conjunction with FIGS. 3 and 4, or includes an active element, as illustrated in and described in conjunction with FIGS. 9 and 10, the methods can include implanting the first intraluminal scaffold at one side of the valve, with the weighted element or the active element disposed on another side of the valve, e.g., downstream of the valve.

In the case of scaffold assemblies including a second intraluminal scaffold coupled to the elongated core member and spaced from the first intraluminal scaffold, as those illustrated in FIGS. 14-15, the delivery method can further include implanting the second intraluminal scaffold at a second target site. The first target site and the second target site can be upstream and downstream of the valve, respectively, or vice versa. In this way, the elongated core member between the first intraluminal scaffold and the second intraluminal scaffold traverses the valve. The first target site and the second target site can be in the same blood vessel or different blood vessels. For example, the first intraluminal scaffold and the second intraluminal scaffold can be both deployed in an internal jugular vein (e.g., the first intraluminal scaffold and the seconds scaffold are disposed at either side of the valve), or be deployed in an internal jugular vein and a brachiocephalic vein, respectively.

Using a delivery system as described above, a delivery process for an intraluminal scaffold assembly including two scaffolds as above is schematically described below. Before delivery, the two scaffolds and the elongated core member can be positioned in the distal portion of the delivery catheter, with the first intraluminal scaffold being positioned more distally than the second intraluminal scaffold. The distal portion of the catheter can then be positioned proximate a second target site where the second intraluminal scaffold is to be implanted. The intraluminal scaffold assembly is then deployed by retracting the outer sheath and/or advancing the inner member of the catheter distally to expose the first intraluminal scaffold at the first target site. The process is repeated or continued, as appropriate to expose the second intraluminal scaffold at the second target site, with the elongated core member traversing a valve of interest. Alternatively, the distal portion of the catheter can be positioned proximate the first target site where the first intraluminal scaffold is to be implanted. The intraluminal scaffold assembly is then deployed by retracting the outer sheath to expose the first intraluminal scaffold and the second intraluminal scaffold out of the outer sheath, thereby implanting the first intraluminal scaffold at the first target site and the second intraluminal scaffold at the second target site. It is appreciated that the connector portion of the elongated core member and/or either portion exposed and extending away from either scaffold can be disposed to traverse the valve.

For delivering the two-scaffold assembly as illustrated in FIGS. 14 and 15 at a bifurcation of blood vessels, the delivery catheter can be steered by a guidewire, e.g., a RX guidewire, or by an enclosing guiding catheter to navigate the branched geometry. If the elongated core member between the two scaffolds includes a portion biased in a non-linear shape, a particular delivery catheter with a flexible tip can be used. The construction and use of such a delivery catheter are illustrated in FIGS. 22A and 22B. As illustrated in FIG. 22A, a delivery catheter 2000 include a distal end having at least two sections of different longitudinal stiffness. The first, more proximal, section 2010 has a greater longitudinal stiffness as compared to a second, more distal, section 2020. These two sections can be formed with varying longitudinal stiffness using a number of techniques. For example, the method of construction of the sections can be varied and/or first section can comprise braiding while the distal section may comprise a coil, in addition, the material and size of the filaments used to construct the braiding or coil may be varied in order to produce the desired stiffness profile. For example, the first section braid can be fabricated using metallic filaments while the second section coil may be fabricated using a polymer filament. It will be appreciated that both sections may be formed using the same type and size of filament material; however, the longitudinal stiffness may be affected by the braid and/or coil pitch in various locations.

The delivery catheter 2000 can be used to deploy an intraluminal scaffold assembly such as the one illustrated in FIG. 14. As shown in FIG. 22A, the intraluminal scaffold assembly can be first placed in the proximal portion 2010 of the catheter and engage the inner member 2080 of the catheter. At this initial position, the proximal section of the catheter 2010 remains straightened because of its greater longitudinal stiffness than the stiffness of the non-linear connector portion 1210 of the intraluminal scaffold assembly. By contrast, the distal section 2020 is flexible enough to allow it bend when the intraluminal scaffold assembly is disposed therein. In FIG. 22B, as the intraluminal scaffold assembly is moved distally by the relative motion between the outer sheath and the inner member 2080, the distal section 2020 begins to curve to take on the shape of the non-linear shape of the connector portion 1210. As such, the stiffness of the connector portion 1210 should be greater than the longitudinal stiffness of the distal portion 2020 so that the distal section 2020 will bend or deform as desired. The catheter tip allows it to be easily steered through a branched anatomy, e.g., simply by rotating and advancing the catheter as needed. In this way, the catheter steering is much like the steering of a guidewire or a guiding catheter, with which the operating physician will be familiar.

For delivering an intraluminal scaffold assembly that includes three interconnected scaffolds, as previously described in connection with FIGS. 16A and 16B, the above delivery method can further include implanting the third intraluminal scaffold at a third target site. The third target site can be located in a blood vessel different from each of the blood vessels in which the first target site and the second target sites are located. In one embodiment, the first target site, the second target site, and the third target site are in an internal jugular vein, brachiocephalic vein, and a subclavian vein, respectively, as shown in FIG. 16B.

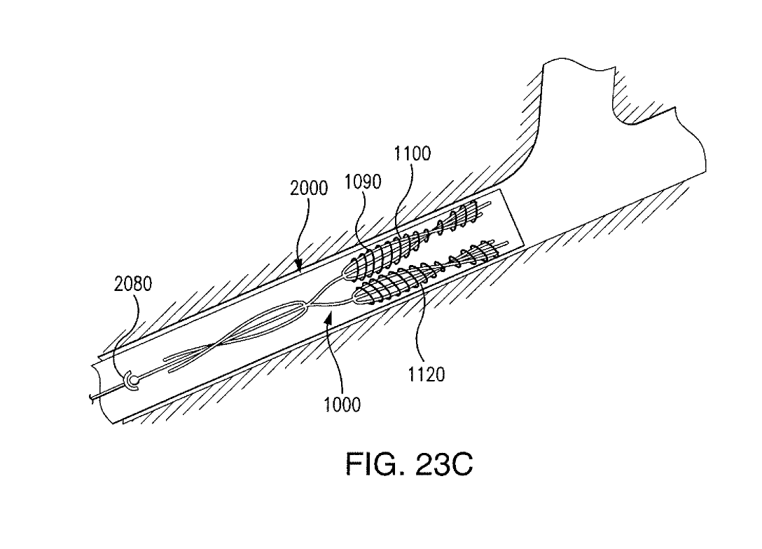

An exemplary delivery process for an intraluminal scaffold assembly including three interconnected scaffolds is illustrated in FIGS. 23A and 23B. In FIG. 23A, the distal end of the catheter 2000 is positioned approximate a bifurcation of blood vessels. The first intraluminal scaffold 1100 and the third intraluminal scaffold 1120, as depicted herein, are placed both distally relative to the second intraluminal scaffold. Furthermore, each scaffold can be collapsed in the delivery configuration can be arranged sequentially along an axis of the delivery catheter, such that each scaffold will be implanted sequentially as the outer sheath is retracted relative to the inner member. That is, and upon retraction of the outer sheath, the first intraluminal scaffold can be deployed upstream of the valve, and the third intraluminal scaffold can then be deployed in the subclavian vein. The outer sheath of the catheter can be further retracted, thereby deploying the second intraluminal scaffold 1110. The elongated core members disposed between the three scaffolds can be pre-bent, e.g., to facilitate the placement of the first intraluminal scaffold into the internal jugular vein.

In addition to the outer sheath of a delivery catheter, other means can be used to control the profile and deployment of one or more of the intraluminal scaffolds. For example, one or more of the intraluminal scaffolds can be constrained in a low profile, as illustrated in FIG. 23C. As embodied herein, the first intraluminal scaffold 1100 and the third intraluminal scaffold 1120 each has a longitudinal axis and includes a plurality of flexible filaments extending from a head portion. The filaments are constrained by a pullwire 1090 wound thereon before deployment. The pullwire can reduce the profile of the intraluminal scaffold(s) while the scaffold is retained in the catheter, as well as prevent entanglement of the filaments between the first and the third intraluminal scaffolds when placed side-by-side in the catheter lumen. To deploy the first intraluminal scaffold 1100 and the third intraluminal scaffold 1120, the scaffolds can be exposed at the distal end position, and then/simultaneously the pullwire 1090 is removed to allow the first intraluminal scaffold 1100 the third intraluminal scaffold 1120 to expand and engage the walls of the respective target vessels.

While illustrative embodiments of the invention have been disclosed herein, numerous modifications and other embodiments may be devised by those skilled in the art in accordance with the invention. For example, the various features depicted and described in the embodiments herein can be altered or combined to obtain desired scaffold characteristics in accordance with the invention. Therefore, it will be understood that the appended claims are intended to include such modifications and embodiments, which are within the spirit and scope of the present invention.

1. An intraluminal scaffold assembly comprising:
a first intraluminal scaffold; and
an elongated core member coupled with the first intraluminal scaffold, the elongated core member having a length sufficient to traverse a valve in a body lumen with the first intraluminal scaffold positioned proximate the valve.

2. The intraluminal scaffold assembly of claim 1, further comprising a weighted element coupled to the elongated core member.

3. The intraluminal scaffold assembly of claim 1, further comprising a second intraluminal scaffold coupled with the elongated core member and spaced from the first intraluminal scaffold.

4. The intraluminal scaffold assembly of claim 3, where at least a portion of the elongated core member between the first intraluminal scaffold and the second intraluminal scaffold is biased in a non-linear shape.

5. The intraluminal scaffold assembly of claim 3, further comprising a second elongated core member extending from the first elongated core member at a connection location between the first intraluminal scaffold and the second intraluminal scaffold, and a third intraluminal scaffold coupled with the second elongated core member.

6. The intraluminal scaffold assembly of claim 5, wherein the first elongated core member and the second elongated core member define a hinge at the connection location.

7. The intraluminal scaffold assembly of claim 1, wherein the first intraluminal scaffold is a supporting scaffold.

8. The intraluminal scaffold assembly of claim 1, wherein the first intraluminal scaffold is a conforming scaffold.

9. The intraluminal scaffold assembly of claim 1, wherein the elongated core member further includes a tail element extending laterally from the elongated core member.

10. The intraluminal scaffold assembly of claim 9, wherein the elongated core member includes at least two tail elements, wherein each of the at least two tail elements extends laterally from the elongated core member.

11. The intraluminal scaffold assembly of claim 10, wherein the at least two tail elements include at least three tail elements, wherein at least two of the at least three tail elements extend from a same location along the elongated core member, and wherein at least one of the at least three tail elements extend from a second location along the elongated core member different than the first location.

12. The intraluminal scaffold assembly of claim 9, wherein the tail element has a profile sufficient to open a valve in a body lumen with the intraluminal scaffold positioned proximate the valve.

13. The intraluminal scaffold assembly of claim 9, wherein the tail element is made of a material different than that of remainder of the elongated core member.

14. The intraluminal scaffold assembly of claim 1, wherein the intraluminal scaffold defines a longitudinal axis and includes at least two filaments extending from a head portion disposed along the longitudinal axis at a first longitudinal end.

15. The intraluminal scaffold assembly of claim 14, wherein the elongated core member is coupled with the intraluminal scaffold at the head portion.

16. The intraluminal scaffold assembly of claim 14, wherein the at least two filaments of the intraluminal scaffold each includes an end portion disposed at a second longitudinal end opposite the head portion.

17. The intraluminal scaffold assembly of claim 14, wherein the end portions of the at least two filaments of the intraluminal scaffold are joined together approximate the second longitudinal end of the intraluminal scaffold.

18. The intraluminal scaffold assembly of claim 17, wherein the elongated core member is coupled with the end portions of the at least two filaments at the second longitudinal end of the intraluminal scaffold.

19. The intraluminal scaffold assembly of claim 1, further comprising:
an active element coupled with the elongated core member at a location spaced from the intraluminal scaffold, the active element being externally actuatable to deflect the elongated core member.

20. The intraluminal scaffold assembly of claim 19, wherein the active element is magnetically active.

21. The intraluminal scaffold assembly of claim 19, further comprising a source of external field to actuate the active element.

22. The intraluminal scaffold assembly of claim 21, wherein the source of external field comprises at least one magnet.

23. The intraluminal scaffold assembly of claim 22, wherein the at least one magnet is disposed within a housing structure.

24. The intraluminal scaffold assembly of claim 23, wherein the housing structure is a pillow or a wearable article.

25. The intraluminal scaffold assembly of claim 22, wherein the external field has at least one of an adjustable strength and an adjustable orientation

26. The intraluminal scaffold assembly of claim 22, further comprising a controller to control the external field according to a preset schedule.

27. An intraluminal scaffold assembly of claim 3, further comprising:
a weighted node coupled to the elongated core member at a location between the first intraluminal scaffold and the second intraluminal scaffold.

28. The intraluminal scaffold assembly of claim 27, wherein the weighted node has a mass sufficient to cause the elongated core member to urge a portion of the valve toward an open position.

29. A method of delivering an intraluminal scaffold assembly, comprising:
providing an intraluminal scaffold assembly, the intraluminal scaffold assembly comprising a first intraluminal scaffold and an elongated core member coupled with the first intraluminal scaffold;
deploying the intraluminal scaffold assembly by implanting the first intraluminal scaffold at a first target site within a body lumen with the elongated core member disposed to cross a valve.

30. The method of claim 29, wherein the first target site is upstream of the valve.

31. The method of claim 29, wherein the body lumen is a blood vessel.

32. The method of claim 31, wherein the first target site is in an internal jugular vein.

33. The method of claim 31, wherein the first target site is in a different blood vessel from a blood vessel housing the valve.

34. The method of claim 31, wherein the intraluminal scaffold assembly further comprises a weighted element coupled to the elongated core member, wherein the first intraluminal scaffold is implanted at one side of the valve with the weighted element disposed on another side of the valve.

35. The method of claim 31, wherein the first intraluminal scaffold is implanted in an external jugular vein with the elongated core member disposed to cross a valve in a neighboring internal jugular vein.

36. The method of claim 29, wherein the intraluminal scaffold assembly further comprises a second intraluminal scaffold coupled to the elongated core member and spaced from the first intraluminal scaffold, and wherein deploying the intraluminal scaffold assembly further comprises implanting the second intraluminal scaffold at a second target site.

37. The method of claim 36, wherein the first target site is upstream of the valve and the second target site is downstream of the valve.

38. The method of claim 36, wherein the first target site is in an internal jugular vein and the second target site is in a brachiocephalic vein.

39. The method of claim 36, wherein a portion of the elongated core member between the first intraluminal scaffold and the second intraluminal scaffold is biased in a non-linear shape.

40. The method of claim 36, disposing the elongated core member to cross at least one valve in at least one of the left internal jugular vein or the right internal jugular vein of the patient.

41. The method of claim 40, wherein the first intraluminal scaffold is implanted in the left external jugular vein.

42. The method of claim 40, wherein at least a portion of the elongated core member is disposed to pass through a brachiocephalic vein.

43. The method of claim 40, wherein the intraluminal scaffold assembly further comprises a weighted node coupled to the elongated core member at a location between the first intraluminal scaffold and the second intraluminal scaffold.

44. The method of claim 43, wherein disposing the elongated core member includes disposing the weighted node proximate the upper end of superior vena cava.

45. The method of claim 36, wherein the intraluminal scaffold assembly further comprises:
a second elongated core member extending from the first elongated core member at a connection location between the first intraluminal scaffold and the second intraluminal scaffold, and
a third intraluminal scaffold coupled with the second elongated core member;
the method further comprising implanting the third intraluminal scaffold at a third target site.

46. The method of claim 45, wherein the first target site, the second target site, and the third target site are in joined internal jugular vein, brachiocephalic vein, and subclavian vein, respectively.

47. The method of claim 29, wherein the elongated core member includes at least one tail element extending laterally from the elongated core member, and wherein deploying the intraluminal scaffold assembly comprises disposing the at least one tail element to open the valve.

48. A method of delivering an intraluminal scaffold assembly, comprising:
providing a system, comprising:
a delivery system having an inner member having a distal end portion and an outer sheath movable relative to the inner member, the outer sheath having a first position to cover the distal end portion of the inner member and a second position to expose the distal end portion of the inner member, and
an intraluminal scaffold assembly comprising a first intraluminal scaffold and an elongated core member coupled with the first intraluminal scaffold, the intraluminal scaffold assembly being disposed at the distal end portion of the inner member;
positioning the delivery system with the distal end portion disposed in a body lumen of a patient;
deploying the intraluminal scaffold assembly by moving the outer sheath to the second position relative to the inner member to implant the first intraluminal scaffold at a first target site with the elongated core member disposed across a valve of the body lumen.

49. The method of claim 48, wherein the intraluminal scaffold assembly further comprises a second intraluminal scaffold coupled with the elongated core'member and spaced from the first intraluminal scaffold,
wherein deploying the intraluminal scaffold assembly comprises moving the outer sheath to the second position relative to the inner member to implant the second intraluminal scaffold at a second target site.

50. The method of claim 49, wherein the elongated core member includes a portion biased in a non-linear shape, and the outer sheath of the delivery system comprises a proximal section having a first longitudinal stiffness and a distal section having a second longitudinal stiffness less than the first longitudinal stiffness, and further wherein the biased portion of the elongated core member has a longitudinal stiffness which is less than the first longitudinal stiffness and greater than the second longitudinal stiffness to bend the distal section of the outer sheath when disposed therein.

51. The method of claim 49, wherein the valve is in an internal jugular vein.

52. The method of claim 49, wherein the intraluminal scaffold assembly further comprises a second elongated core member extending from the first elongated core member at a connection location between the first intraluminal scaffold and the second intraluminal scaffold, and a third intraluminal scaffold coupled with the second elongated core member;
wherein deploying the intraluminal scaffold assembly comprises moving the outer sheath to the second position relative to the inner member to further implant the third intraluminal scaffold at a third target site.

53. The method of claim 52, wherein the first target site, the second target site, and the third target site are in joined internal jugular vein, brachiocephalic vein and subclavian vein, respectively.

54. The method of claim 48, wherein the first intraluminal scaffold defines a longitudinal axis and include a plurality of flexible filaments extending from a head portion, the filaments being constrained by a pullwire wound thereon before deployment,
wherein deploying the intraluminal scaffold assembly further comprises removing the pullwire.

55. A system, comprising:
a delivery system having an inner member having a distal end portion and an outer sheath movable relative to the inner member, the outer sheath having a first position to cover the distal end portion of the inner member and a second position to expose the distal end portion of the inner member;
an intraluminal scaffold assembly comprising a first intraluminal scaffold and an elongated core member coupled with the first intraluminal scaffold, the elongated core member having a length sufficient to traverse a valve in a body lumen with the intraluminal scaffold positioned proximate the valve; and
wherein the intraluminal scaffold assembly releasably engages a distal end of the inner member of the delivery system.

56. The system of claim 55, wherein the first intraluminal scaffold defines a longitudinal axis and includes a plurality of flexible filaments extending from a head portion disposed along the longitudinal axis at a first longitudinal end, each of the at least two filaments including a free end portion at a second longitudinal end opposite the head portion, the at least two filaments converging toward each other at a juncture disposed proximate the longitudinal axis between the first longitudinal end and the second longitudinal end.

57. The system of claim 55, wherein the intraluminal scaffold assembly further includes a second intraluminal scaffold coupled with the elongated core member and spaced from the first intraluminal scaffold.

58. The system of claim 57, wherein the elongated core member includes a portion biased in a non-linear shape, and the outer sheath of the delivery system comprises a proximal section having a first longitudinal stiffness and a distal section having a second longitudinal stiffness less than the first longitudinal stiffness, and further wherein the biased portion of the elongated core member has a longitudinal stiffness which is less than the first longitudinal stiffness and greater than the second longitudinal stiffness to bend the distal section of the outer sheath when disposed therein.

59. The system of claim 57, wherein the intraluminal scaffold assembly further includes a second elongated core member extending from the first elongated core member at a connection location between the first intraluminal scaffold and the second intraluminal scaffold, and a third intraluminal scaffold coupled with the second elongated core member.

60. A method of manipulating a valve of a body lumen of a patient, comprising:
providing an intraluminal scaffold assembly including an intraluminal scaffold, an elongated core member coupled with the intraluminal scaffold, and an active element coupled to the elongated core member and spaced from the intraluminal scaffold;
deploying the intraluminal scaffold assembly in a body lumen of a patient by implanting the intraluminal scaffold at a target site with the elongated core member disposed to cross a valve of the body lumen; and
applying an external field to actuate the active element to cause a deflection of the elongated core member, thereby causing a disturbance of the valve.

61. The method of claim 60, wherein the body lumen is a blood vessel.

62. The method of claim 61, wherein the blood vessel is an internal jugular vein.

63. The method of claim 61, wherein the target site is in a second blood vessel different from the blood vessel housing the valve.

64. The method of claim 63, wherein the second blood vessel is the external jugular vein.

65. The method of claim 60, wherein deploying the intraluminal scaffold assembly includes implanting the intraluminal scaffold on one side of the valve and disposing the active element on another side of the valve.

66. The method of claim 60, wherein the external field is applied when the patient is in a supine or prone position.

67. The method of claim 60, wherein applying the external field comprises controlling a strength, orientation, or a combination thereof of the external field.
